(19) United States
(12) Patent Application Publication
(10) Pub. No.: US 2016/0235717 A1
(43) Pub. Date: Aug. 18, 2016

(54) BENDAMUSTINE PHARMACEUTICAL COMPOSITIONS

(71) Applicant: LUITPOLD PHARMACEUTICALS, INC., Shirley, NY (US)

(72) Inventors: Gopal Anyarambhatla, New Albany, OH (US); Isaac Agyemang, New Albany, OH (US); William C. Schumacher, New Albany, OH (US); Jerry Zhang, New Albany, OH (US); Ashvin Patel, New Albany, OH (US)

(21) Appl. No.: 15/026,832

(22) PCT Filed: Oct. 9, 2014
(86) PCT No.: PCT/US2014/059983

§ 371 Date: Apr. 1, 2016

Related U.S. Application Data
(60) Provisional application No. 61/889,950, filed on Oct. 11, 2013.

(51) Int. Cl. A61K 31/4184; A61K 9/19; A61K 47/26; A61K 9/08; A61K 47/20

(52) U.S. Cl. CPC A61K 31/4184; A61K 9/08; A61K 47/20; A61K 47/26; A61K 9/19

(57) ABSTRACT

Pharmaceutical formulations of bendamustine for pharmaceutical use and methods related thereto. The formulations and methods described herein utilize a polar aprotic solvent, or mixture thereof, that can be used to produce lyophilized bendamustine.

### CROSS-REFERENCE TO RELATED APPLICATIONS

The present application claims the benefit of U.S. Provisional Application Ser. No. 61/889,950 filed 11 Oct. 2013, which is incorporated herein by reference in its entirety.

### BACKGROUND OF THE INVENTION

Treanda® is the bulk composition of the active pharmaceutical ingredient (API), bendamustine hydrochloride, mannitol, and solvent (U.S. Pat. No. 8,436,190, U.S. Pat. No. 8,445,524, incorporated herein by reference). Tertiary Butyl Alcohol (TBA) and other organic solvents can be used as the organic solvent during bulk preparation which can be later removed along with water by lyophilization to obtain the finished product. Other formulations using bendamustine are disclosed in WO 2012/103226, incorporated herein by reference.

The salt bendamustine hydrochloride is an alkylating agent, originally synthesized in 1963 at the Institute for Microbiology & Experimental Therapy in Jena, German Democratic Republic, with the intent to produce an agent with both alkylating and antimetabolite properties. Jenapharm (now Schering AG) formerly marketed it in Germany under the trade name Cytostasan from 1971 to 1992. Cytostasan was a lyophilized powder for solution for injection (vials) containing 25 mg of bendamustine HCl. It was widely used but never studied systematically in patients until the 1990s, then German investigators demonstrated its clinical activity in a number of malignancies. Since 1993, Ribosepharm was marketing bendamustine in Germany under the brand name Ribomustin RBO. Ribomustin is available as a lyophilized powder for injection, containing 100 mg of drug in each 50 ml vial, or 25 mg of drug in each 20 ml vial, also comprising mannitol, and indicated for the treatment of chronic lymphocytic leukemia. It was shown that the lyophilized powder should be reconstituted as close to the time of patient administration as possible with 40 ml (for a 100 mg product) or 10 ml (for a 25 mg product) of sterile water for injection. The reconstituted product was then further diluted to 500 ml with 0.9% sodium chloride for injection. The route of administration was by intravenous infusion over 30 to 60 minutes.

Bendamustine Hydrochloride for Injection is also sold in the United States by Cephalon, Inc. as Treanda® for Injection, a lyophilized powder in a single-use vial indicated for the treatment of patients with chronic lymphocytic leukemia and indolent B-cell non-Hodgkin's lymphoma. A 25 mg dose vial contains 25 mg of bendamustine hydrochloride and 42.5 mg of mannitol, and a 100 mg dose vial contains 100 mg of bendamustine hydrochloride and 170 mg of mannitol.

Treanda® is intended for intravenous infusion only after reconstitution with sterile water for injection USP, and then further dilution with either 0.9% Sodium Chloride Injection, USP, or 2.5% Dextrose/0.45% Sodium Chloride Injection, USP. The pH of the reconstituted solution is 2.5-3.5. Treanda® is supplied as a sterile non-pyrogenic white to off-white lyophilized powder, in a single-use vial.

Bendamustine hydrochloride has been shown to be unstable in an aqueous solution. Due to bendamustine degradation in aqueous solutions (similar to other nitrogen mustards), bendamustine is generally provided in lyophilized products. The finished lyophilizate can be unstable when exposed to light. Therefore, the product frequently can be stored in brown or amber-colored glass bottles. The lyophilized formulation of bendamustine can contain degradation products that may occur during manufacturing of the drug substance and/or during the lyophilization process to make the finished drug product. Moreover, reconstitution of the lyophilized powder can be difficult and the reconstitution time depends on the solvent used during lyophilization and the manufacturing parameters. Reports from clinical experience indicate that reconstitution can require at least fifteen minutes and may require as long as thirty minutes. In addition to being troublesome and time-consuming for the healthcare professional responsible for reconstituting the product, the lengthy exposure of bendamustine to water during the reconstitution process increases the potential for loss of potency and impurity formation, due to hydrolysis of the product by water.

Because of the high reactivity of nitrogen mustards in aqueous solutions, nitrogen mustards can be difficult to formulate as pharmaceuticals and can be often supplied for administration in a lyophilized form that requires reconstitution, usually in water, by skilled hospital personnel prior to administration. Once in aqueous solution, nitrogen mustards can be subject to degradation by hydrolysis; thus, the reconstituted product should be administered to a patient as soon as possible after its reconstitution and reconstitution time should be short enough to reduce degradation.

A lyophilized powder can contain bendamustine HCl and suitable bulking agent. For example, one of the suitable bulking agents can be mannitol. It has been previously shown that mannitol cannot be dissolved in completely organic solvent systems. Therefore, water should be a part of solvent system for preparing bulk solution. To lower the rate of degradation of bendamustine HCl, solvent systems for manufacturing bulk solution, the sequence of addition of ingredients, temperatures, duration of steps in lyophilization, and the like can be important.

German Democratic Republic (GDR) Patent No. 34727 discloses a method of preparing -[5-bis-(beta-chloroethyl)-amino-benzimidazolyl-(2)]-alkane carboxylic acids that are substituted in the 1-position. GDR Patent No. 80967 discloses an injectable preparation of y-[1-methyl-5-bis-(beta-chloroethyl)-amino-benzimaidazolyl-(2)-]-butric acid hydrochloride. GDR Patent No. 159877 discloses a method for preparing 4-[1-methyl-5-bis(2-chloroethyl) amino-benzimidazolyl-2)-butyric acid. GDR Patent No. 159289 discloses an injectable solution of bendamustine. An article by N. Ni et al., “Use of Pure t-Butanol as a Solvent for Freeze-Drying: a Case Study,” International Journal of Pharmaceutics, Vol. 226, Issues 1-2, pages 39-46 (2001), reports that 1-(2-chloroethyl)-3-sarcosinamide-1-nitrosourea was more stable in pure tertiary-butanol (TBA) than in pure acetic acid, dimethylsulfoxide, water, or in TBA-water mixtures. Lyophilized cyclophoshamide is disclosed in U.S. Pat. Nos. 5,418,223, 5,413,995, 5,268,368, 5,227,374, 5,130,305, 4,659,699, 4,537,883, and 5,066,647. The lyophilized nitrogen mustard compound ifosfamide is disclosed in International Application Publication No. WO 2003/066027 and U.S. Pat. Nos. 6,613,927, 5,750,131, 5,972,912, 5,227,373, and 5,204,335. Lyophilized formulations of prostaglandin E-1, made by dissolving PGE-1 in a solution of lactose and tertiary-butyl alcohol, are disclosed in U.S. Pat. No. 5,770,230. U.S. Patent Application Publication No. 2006/0159713 discloses pharmaceutical formulations of lyophilized bendamustine, suitable for pharmaceutical use, and provides methods of producing lyophilized bendamustine. The pharmaceutical formulations can be used for any disease that is sensitive to treatment with bendamustine, such as neoplastic diseases.

### SUMMARY OF THE INVENTION

Among the various aspects of the present disclosure is the provision of a bendamustine formulation for lyophilization that can include a stabilizing concentration of a polar aprotic solvent (e.g., DMSO) and an optional excipient (e.g., mannitol).

One aspect of the present disclosure provides a pre-lyophilization solution. In some embodiments, the pre-lyophilization solution includes bendamustine or a pharmaceutically acceptable salt thereof; a pharmaceutically acceptable excipient; and an organic solvent comprising dimethyl sulfoxide (DMSO).

In some embodiments, the pre-lyophilization solution comprises about 40% to about 100% organic solvent. In some embodiments, the pre-lyophilization solution comprises about 40% to about 100% dimethyl sulfoxide (DMSO). In some embodiments, the pre-lyophilization solution comprises about 80% to about 100% organic solvent. In some embodiments, the pre-lyophilization solution comprises about 40% to about 100% dimethyl sulfoxide (DMSO). In some embodiments, the pre-lyophilization solution comprises about 100% organic solvent. In some embodiments, the pre-lyophilization solution comprises about 100% dimethyl sulfoxide (DMSO).

In some embodiments, the bendamustine is present at a concentration of about 1 mg/ml to about 85 mg/ml. In some embodiments, the bendamustine is present at a concentration of about 15 mg/ml to about 50 mg/ml.

In some embodiments, the bendamustine comprises a Form 2 bendamustine polymorph.

In some embodiments, the pharmaceutically acceptable excipient comprises one or more of sodium phosphate, potassium phosphate, citric acid, tartaric acid, gelatin, glycine, and carbohydrates such as lactose, sucrose, maltose, mannitol, glycerin, dextrose, dextran, trehalose, or hetastarch or a mixture thereof. In some embodiments, the pharmaceutically acceptable excipient comprises mannitol. In some embodiments, the pharmaceutically acceptable excipient comprises mannitol at a concentration of about 25 mg/ml to about 85 mg/ml.

In some embodiments, the pharmaceutically acceptable salt is selected from the group consisting of 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; hydrobromic acid; hydrochloric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; nitric acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid; or a mixture thereof. In some embodiments, the pharmaceutically acceptable salt is HCl.

In some embodiments, the organic solvent does not comprise tert-butyl alcohol.

Another aspect provides a method for preparing a solution described above. In some embodiments, the method includes combining bendamustine or a pharmaceutically acceptable salt thereof, a pharmaceutically acceptable excipient, and the organic solvent.

Another aspect provides a method for compounding a bendamustine pharmaceutical composition. In some embodiments, the method includes combining bendamustine or a pharmaceutically acceptable salt thereof, a pharmaceutically acceptable excipient, and an organic solvent to form a solution described above, and lyophilizing such solution to form a lyophilized bendamustine composition.

In some embodiments, the lyophilized bendamustine composition comprises water at a concentration of about 0% to about 2% by weight. In some embodiments, the lyophilized bendamustine composition comprises water at a concentration of about 0%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2% by weight.

In some embodiments, the method further includes reconstituting the lyophilized bendamustine composition to form a bendamustine pharmaceutical composition suitable for administration to a subject. In some embodiments, the time required for reconstitution is less than about 300 seconds. In some embodiments, the time required for reconstitution is less than about 120 seconds. In some embodiments, the time required for reconstitution is less than about less than about 300 seconds, less than about 270 seconds, less than about 240 seconds, less than about 210 seconds, less than about 180 seconds, less than about 150 seconds, less than about 120 seconds, less than about 90 seconds, less than about 60 seconds, less than about 50 seconds, less than about 40 seconds, less than about 30 seconds, less than about 25 seconds, less than about 20 seconds, less than about 15 seconds, or less than about 10 seconds.

In some embodiments, a lyophilized bendamustine composition or the bendamustine pharmaceutical composition comprises less than about 8% bendamustine-related impurities. In some embodiments, a lyophilized bendamustine composition or the bendamustine pharmaceutical composition comprises less than about 1.0%, less than about 0.9%, less than about 0.8%, less than about 0.7%, less than about 0.6%, less than about 0.5%, less than about 0.4%, less than about 0.3%, less than about 0.25%, less than about 0.2%, less than about 0.15%, less than about 0.1%, or less than about 0.05% bendamustine-related impurities. In some embodiments, a lyophilized bendamustine composition or the bendamustine pharmaceutical composition comprises less than about 0.25% bendamustine-related impurities.

Another aspect provides a pharmaceutical composition comprising bendamustine prepared according to a method described above. In some embodiments, the composition comprises less than 1% impurities.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a scatter plot depicting the freezing point of DMSO-water mixtures (Dimethyl Sulfoxide (DMSO) literature, Arkema).

FIG. 2 is a screenshot of the freeze-drying parameters for a Virtis Model (Genesis 25 xL) used for the lyophilization for samples described in Examples 1-8.

FIG. 3 is the Liquid Chromatography Chromatogram for the initial bulk solution sample 6IB130708-1 using LC sequence 070813.

FIG. 4 is the Liquid Chromatography (LC) Chromatogram for the bulk solution at 17 hours at room temperature sample 6IB130708-1 using LC sequence 070813.

FIG. 5 is the Liquid Chromatography (LC) Chromatogram for the final product, 100 mg/vial sample 6IB130708-1 using LC sequence 071513.

FIG. 6 is the Liquid Chromatography (LC) Chromatogram for the initial bulk solution sample 6IB130708-2 using LC sequence 070813.

FIG. 7 is the Liquid Chromatography (LC) Chromatogram for the bulk sample 6IB130708-2 at 17 hours at 15° C. using LC sequence 070813.

FIG. 8 is the Liquid Chromatography (LC) Chromatogram for the final product, 100 mg/vial sample 6IB130708-2 using LC sequence 071513.

FIG. 9 is the Liquid Chromatography (LC) Chromatogram for the initial bulk solution sample 6IB130709-2 using LC sequence 070813.

FIG. 10 is the Liquid Chromatography (LC) Chromatogram for the bulk solution sample 6IB130709-2 at 20 hours at 15° C. using LC sequence 070813.

FIG. 11 is the Liquid Chromatography (LC) Chromatogram for the bulk solution sample 6IB130709-2 at 20 hours at 5° C. using LC sequence 070813.

FIG. 12 is the Liquid Chromatography (LC) Chromatogram for the final product, 100 mg/vial sample 6IB130709-2 using LC sequence 071513.

FIG. 13 is a screenshot of the freeze-drying parameters for a Virtis Model (Genesis 25 XL) used for the lyophilization for samples described in Examples 10-16.

FIG. 14 is a graph of the lyophilization time vs. temperature plot

FIG. 15 is the Liquid Chromatography (LC) Chromatogram for the initial bulk solution sample 2JZ130726P32 using LC sequence 072513.

FIG. 16 is the Liquid Chromatography (LC) Chromatogram for the initial bulk solution sample 2JZ130726P31-15 using LC sequence 072513.

FIG. 17 is the Liquid Chromatography (LC) Chromatogram for the initial bulk solution sample 2JZ130726P31-25 using LC sequence 072513.

FIG. 18 is the Liquid Chromatography (LC) Chromatogram for the T1, room temperature bulk solution sample 2JZ130726P32 using LC sequence 072513.

FIG. 19 is the Liquid Chromatography (LC) Chromatogram for the T1, 5° C. sample 2JZ130726P32 using LC sequence 072513.

FIG. 20 is the Liquid Chromatography (LC) Chromatogram for the T1, room temperature bulk solution sample 2JZ130726P31-15 using LC sequence 072513.

FIG. 21 is the Liquid Chromatography (LC) Chromatogram for the T1, room temperature bulk solution sample 2JZ130726P31-25 using LC sequence 072513.

FIG. 22 is the Liquid Chromatography (LC) Chromatogram for the initial, 100 mg/vial final product sample 2JZ130726P16 using LC sequence 072513.

FIG. 23 is the Liquid Chromatography (LC) Chromatogram for the 100 mg/vial (Lot TA30912) sample reference listed drug (RLD), using LC sequence 072513.

FIG. 24 is the Liquid Chromatography (LC) Chromatogram for the initial 100 mg/vial final product reconstitution sample 2JZ130726P16 using LC sequence 072513.

FIG. 25 is the Liquid Chromatography (LC) Chromatogram for the 0.2 mg/ml DMSO standard prep sample using LC sequence 072913.

FIG. 26 is the Liquid Chromatography (LC) Chromatogram for the 100 mg/vial final product sample 2JZ130726P16 using LC sequence 072913.

### DETAILED DESCRIPTION OF THE INVENTION

The present disclosure is based, at least in part, on the discovery that lyophilization using DMSO can stabilize bendamustine against hydrolysis or other related degradation mechanisms or decrease time required for reconstitution of the lyophilized bendamustine composition. Bendamustine formulations can degrade in aqueous solutions, which may be present during product manufacturing, lyophilization, or following reconstitution for dilution/administration. Incorporating certain organic solvent(s) in the pre-lyophilization formulation can help stabilize bendamustine against hydrolysis and other related degradation mechanisms. As disclosed herein, bendamustine formulations for lyophilization can include a stabilizing concentration of a polar aprotic solvent (e.g., DMSO) and an optional excipient (e.g., mannitol).

Formulation protocols, characterization of, and methods of treatment using bendamustine compositions processes are well known; see e.g. U.S. Pat. No. 8,445,524, U.S. Pat. No. 8,436,190, and International Application No. PCT/US2012/022561 (Published as WO 2012/103226) (each of which is incorporated herein by reference in its entirety). Except as otherwise noted herein, therefore, the compositions and process of the present disclosure can be carried out in accordance with such processes.

Organic Solvent

As described herein, bendamustine can be compounded or formulated with an organic solvent. For example, a pre-lyophilization bendamustine solution or a lyophilized bendamustine composition can include bendamustine and an organic solvent, such as dimethylsulfoxide (DMSO).

An organic solvent, as described herein, can include an organic material, usually a liquid, that can be capable of dissolving other substances. For example, organic solvents that can be used include, without limitation thereto, acetone, acetonitrile, benzyl alcohol, 1-pentanol, anisole, carbon tetrachloride, chlorobutanol, cyclohexane, diethyl ether, dimethylamine, dimethylformamide (DMF), dimethylsulfone, dimethylsulfoxide (DMSO), dimethyl carbonate, dichloromethane, dioxane, di-isopropyl ether, ethanol, ethyl acetate, formamide, heptane, hexafluoroacetone, isopropanol (isopropyl alcohol), isopropyl acetate, methyl acetate, methyl ethyl ketone, methyl isobutyl ketone, 1-pentanol, methyl acetate, methanol, nitromethane, N,N-dimethylacetamide, n-propanol, n-butanol, tert-butyl methyl ether, tert-butyl alcohol (TBA), tetrahydrofuran, toluene, or a mixture of two or more thereof. In some embodiments, an organic solvent can include dimethyl sulfoxide (DMSO).

In some embodiments, the organic solvent is not TBA.

As described herein, a composition comprising an organic solvent can be diluted with water or aqueous solutions (e.g., acetic acid) to form a solution for using in compounding or lyophilizing bendamustine.

A compounding or pre-lyophilization bendamustine solution can include at least about 30% organic solvent. For example, a compounding or pre-lyophilization bendamustine solution can include (e.g., v/v) at least about 30% organic solvent; at least about 31% organic solvent; at least about 32% organic solvent; at least about 33% organic solvent; at least about 34% organic solvent; at least about 35% organic solvent; at least about 36% organic solvent; at least about 37% organic solvent; at least about 38% organic solvent; at least about 39% organic solvent; at least about 40% organic solvent; at least about 41% organic solvent; at least about 42% organic solvent; at least about 43% organic solvent; at least about 44% organic solvent; at least about 45% organic solvent; at least about 46% organic solvent; at least about 47% organic solvent; at least about 48% organic solvent; at least about 49% organic solvent; at least about 50% organic solvent; at least about 51% organic solvent; at least about 52% organic solvent; at least about 53% organic solvent; at least about 54% organic solvent; at least about 55% organic solvent; at least about 56% organic solvent; at least about 57% organic solvent; at least about 58% organic solvent; at least about 59% organic solvent; at least about 60% organic solvent; at least about 61% organic solvent; at least about 62% organic solvent; at least about 63% organic solvent; at least about 64% organic solvent; at least about 65% organic solvent; at least about 66% organic solvent; at least about 67% organic solvent; at least about 68% organic solvent; at least about 69% organic solvent; at least about 70% organic solvent; at least about 71% organic solvent; at least about 72% organic solvent; at least about 73% organic solvent; at least about 74% organic solvent; at least about 75% organic solvent; at least about 76% organic solvent; at least about 77% organic solvent; at least about 78% organic solvent; at least about 79% organic solvent; at least about 80% organic solvent; at least about 81% organic solvent; at least about 82% organic solvent; at least about 83% organic solvent; at least about 84% organic solvent; at least about 85% organic solvent; at least about 86% organic solvent; at least about 87% organic solvent; at least about 88% organic solvent; at least about 89% organic solvent; at least about 90% organic solvent; at least about 91% organic solvent; at least about 92% organic solvent; at least about 93% organic solvent; at least about 94% organic solvent; at least about 95% organic solvent; at least about 96% organic solvent; at least about 97% organic solvent; at least about 98% organic solvent; at least about 99% organic solvent; or at least about 100% organic solvent. It is understood that recitation of the above discrete values includes a range between each recited value. A solution discussed above can be used to form a pre-lyophilization formulation or can be used for compounding or lyophilizing bendamustine.

As another example, a compounding or pre-lyophilization bendamustine solution can include about 30% organic solvent; about 31% organic solvent; about 32% organic solvent; about 33% organic solvent; about 34% organic solvent; about 35% organic solvent; about 36% organic solvent; about 37% organic solvent; about 38% organic solvent; about 39% organic solvent; about 40% organic solvent; about 41% organic solvent; about 42% organic solvent; about 43% organic solvent; about 44% organic solvent; about 45% organic solvent; about 46% organic solvent; about 47% organic solvent; about 48% organic solvent; about 49% organic solvent; about 50% organic solvent; about 51% organic solvent; about 52% organic solvent; about 53% organic solvent; about 54% organic solvent; about 55% organic solvent; about 56% organic solvent; about 57% organic solvent; about 58% organic solvent; about 59% organic solvent; about 60% organic solvent; about 61% organic solvent; about 62% organic solvent; about 63% organic solvent; about 64% organic solvent; about 65% organic solvent; about 66% organic solvent; about 67% organic solvent; about 68% organic solvent; about 69% organic solvent; about 70% organic solvent; about 71% organic solvent; about 72% organic solvent; about 73% organic solvent; about 74% organic solvent; about 75% organic solvent; about 76% organic solvent; about 77% organic solvent; about 78% organic solvent; about 79% organic solvent; about 80% organic solvent; about 81% organic solvent; about 82% organic solvent; about 83% organic solvent; about 84% organic solvent; about 85% organic solvent; about 86% organic solvent; about 87% organic solvent; about 88% organic solvent; about 89% organic solvent; about 90% organic solvent; about 91% organic solvent; about 92% organic solvent; about 93% organic solvent; about 94% organic solvent; about 95% organic solvent; about 96% organic solvent; about 97% organic solvent; about 98% organic solvent; about 99% organic solvent; or about 100% organic solvent. It is understood that recitation of the above discrete values includes a range between each recited value. A solution discussed above can be used to form a pre-lyophilization formulation or can be used for compounding or lyophilizing bendamustine.

A desirable solvent can form a stable solution with bendamustine and not appreciably or substantially degrade or deactivate the drug. Solubility of bendamustine in a solvent can be high enough to form a commercially useful concentration of the drug in solvent. A solvent can be capable of being removed easily from an aqueous dispersion or solution of the drug product, e.g., through vacuum drying/lyophilization.

Dimethyl Sulfoxide (DMSO).

Various embodiments of the present disclosure utilize DMSO during compounding or lyophilization of bendamustine, which can stabilize bendamustine against hydrolysis or other related degradation mechanisms or decrease time required for reconstitution of a lyophilized bendamustine composition. As shown herein, incorporation of DMSO (or mixtures thereof) into the pre-lyophilization formulation or during the compounding process, can stabilize bendamustine against degradation for a period of time that can exceed 17 hours and decrease reconstitution time (e.g., less than 30 seconds).

The solubility of bendamustine in DMSO has been reported as 79 mg/ml at 25° C. and bendamustine in water at 25° C. as 15 mg/ml. The freezing point of DMSO has been reported as about 18.2° C. Water and co-solvents can decrease the freezing point. DMSO has a relatively high boiling point of 189° C., which can make it evaporate slowly at atmospheric pressure. DMSO can dissolve a wide range of organic and inorganic substances and can be miscible with many common organic solvents such as alcohol, ethers, chlorinated solvents, or aromatics.

There are various advantages to using an elevated amount of DMSO for compounding or lyophilization of bendamustine.

Incorporation of elevated amounts of DMSO (e.g., greater than 80%, 85%, 90%, 95%, or 100%) in the pre-lyophilization formulation can allow for room temperature compounding or storage. Room temperature compounding or storage can be convenient but is typically not feasible for an aqueous solution or most aqueous/organic mixtures containing bendamustine. For example, use of 100% DMSO can provide at least 72 hours of total bulk solution stability at room temperature. By comparison, the preferred Treanda® formulation degraded after the same time period, even when controlling the bulk solution to low temperatures. As such, results reported herein are surprising.

Use of DMSO (e.g., greater than 30%, 35%, 40%, 45%, 50%, 55%, 60%, 65%, 70%, 75%, 80%, 85%, 90%, 95%, or 100%) at room temperature can provide increased solubility of formulations such as bendamustine (e.g., bendamustine hydrochloride) and, optionally, mannitol (e.g., mannitol USP) without sacrificing product purity. This can allow for reduced compounding times, which can lead to lower total impurities in a pre/post-lyophilization formulation. Increased bulk solution concentrations can lead to shorter lyophilization cycle times, reduced operating costs, or added safety benefits in the finished product (e.g., due to potentially lower residual solvent levels). Experiments herein show that formulations containing up to 50 mg/ml bendamustine hydrochloride in DMSO at room temperature can be attainable.

In contrast, U.S. Pat. No. 8,436,190B2 discloses the solubility limit of bendamustine hydrochloride in the preferred Treanda® formulation was only 27 mg/ml at 5° C. While higher reference listed drug (RLD) bulk solution concentrations could be achieved by increasing the solution temperature (e.g., from 5° C. to room temperature), doing so can render the Treanda® formulation bulk solution unstable. As described herein, bendamustine can be formulated at or with room temperature, increased bulk solution concentration, shorter lyophilization cycle times, or reduced impurities, which in view of Treanda® formulation issues are all surprising results.

Furthermore, DMSO is a Class 3 solvent with known toxicology sufficient to classify the Permissible Daily Exposure (PDE) of the solvent. In contrast to embodiments of the present disclosure, the Treanda® formulation utilizes tert-butyl alcohol (TBA). TBA is not listed in ICH Q3C, and thus, inadequate toxicological data exists to classify the PDE of TBA.

As described herein, a composition comprising DMSO can be diluted with water or an aqueous solution (e.g., acetic acid) to form a solution for using in compounding or lyophilizing bendamustine.

A compounding or pre-lyophilization bendamustine solution can include (e.g., v/v) at least about 30% DMSO. For example, a compounding or pre-lyophilization bendamustine solution can include (e.g., v/v) about 30% to about 100% DMSO. As another example, a compounding or pre-lyophilization bendamustine solution can include (e.g., v/v) about 40% to about 100% DMSO; about 50% to about 100% DMSO; about 60% to about 100% DMSO; about 70% to about 100% DMSO; about 80% to about 100% DMSO; about 90% to about 100% DMSO; or about 95% to about 100% DMSO. A solution discussed above can be used to form a pre-lyophilization formulation or can be used for compounding or lyophilizing bendamustine.

A compounding or pre-lyophilization bendamustine solution can include (e.g., v/v) at least about 30% DMSO. For example, a compounding or pre-lyophilization bendamustine solution can include (e.g., v/v) at least about 30% DMSO; at least about 31% DMSO; at least about 32% DMSO; at least about 33% DMSO; at least about 34% DMSO; at least about 35% DMSO; at least about 36% DMSO; at least about 37% DMSO; at least about 38% DMSO; at least about 39% DMSO; at least about 40% DMSO; at least about 41% DMSO; at least about 42% DMSO; at least about 43% DMSO; at least about 44% DMSO; at least about 45% DMSO; at least about 46% DMSO; at least about 47% DMSO; at least about 48% DMSO; at least about 49% DMSO; at least about 50% DMSO; at least about 51% DMSO; at least about 52% DMSO; at least about 53% DMSO; at least about 54% DMSO; at least about 55% DMSO; at least about 56% DMSO; at least about 57% DMSO; at least about 58% DMSO; at least about 59% DMSO; at least about 60% DMSO; at least about 61% DMSO; at least about 62% DMSO; at least about 63% DMSO; at least about 64% DMSO; at least about 65% DMSO; at least about 66% DMSO; at least about 67% DMSO; at least about 68% DMSO; at least about 69% DMSO; at least about 70% DMSO; at least about 71% DMSO; at least about 72% DMSO; at least about 73% DMSO; at least about 74% DMSO; at least about 75% DMSO; at least about 76% DMSO; at least about 77% DMSO; at least about 78% DMSO; at least about 79% DMSO; at least about 80% DMSO; at least about 81% DMSO; at least about 82% DMSO; at least about 83% DMSO; at least about 84% DMSO; at least about 85% DMSO; at least about 86% DMSO; at least about 87% DMSO; at least about 88% DMSO; at least about 89% DMSO; at least about 90% DMSO; at least about 91% DMSO; at least about 92% DMSO; at least about 93% DMSO; at least about 94% DMSO; at least about 95% DMSO; at least about 96% DMSO; at least about 97% DMSO; at least about 98% DMSO; at least about 99% DMSO; or at least about 100% DMSO. It is understood that recitation of the above discrete values includes a range between each recited value. A solution discussed above can be used to form a pre-lyophilization formulation or can be used for compounding or lyophilizing bendamustine.

As another example a compounding or pre-lyophilization bendamustine solution can include (e.g., v/v) about 30% DMSO; about 31% DMSO; about 32% DMSO; about 33% DMSO; about 34% DMSO; about 35% DMSO; about 36% DMSO; about 37% DMSO; about 38% DMSO; about 39% DMSO; about 40% DMSO; about 41% DMSO; about 42% DMSO; about 43% DMSO; about 44% DMSO; about 45% DMSO; about 46% DMSO; about 47% DMSO; about 48% DMSO; about 49% DMSO; about 50% DMSO; about 51% DMSO; about 52% DMSO; about 53% DMSO; about 54% DMSO; about 55% DMSO; about 56% DMSO; about 57% DMSO; about 58% DMSO; about 59% DMSO; about 60% DMSO; about 61% DMSO; about 62% DMSO; about 63% DMSO; about 64% DMSO; about 65% DMSO; about 66% DMSO; about 67% DMSO; about 68% DMSO; about 69% DMSO; about 70% DMSO; about 71% DMSO; about 72% DMSO; about 73% DMSO; about 74% DMSO; about 75% DMSO; about 76% DMSO; about 77% DMSO; about 78% DMSO; about 79% DMSO; about 80% DMSO; about 81% DMSO; about 82% DMSO; about 83% DMSO; about 84% DMSO; about 85% DMSO; about 86% DMSO; about 87% DMSO; about 88% DMSO; about 89% DMSO; about 90% DMSO; about 91% DMSO; about 92% DMSO; about 93% DMSO; about 94% DMSO; about 95% DMSO; about 96% DMSO; about 97% DMSO; about 98% DMSO; about 99% DMSO; or about 100% DMSO. It is understood that recitation of the above discrete values includes a range between each recited value. A solution discussed above can be used to form a pre-lyophilization formulation or can be used for compounding or lyophilizing bendamustine.

It has been reported that the excipient mannitol (e.g., used as a bulking agent) cannot be dissolved in a completely organic solvent system and, thus, requires water be part of a solvent system for any bendamustine formulation (WO2012/103226, page 4). In contrast to these teachings, the present disclosure demonstrates that mannitol can in fact be used with 100% DMSO or other elevated levels of DMSO. This is a surprising result in light of prior reports.

DMSO can be used in combination with one or more other organic solvents described herein.

Bendamustine

As described herein, bendamustine can be compounded or formulated with an organic solvent, such as DMSO, which can stabilize bendamustine against hydrolysis or other related degradation mechanisms.

Bendamustine is the international nonproprietary name (INN) for a compound having a chemical names including: 4-[5-[Bis(2-chloroethyl)amino]-1-methylbenzimidazol-2-yl]butanoic acid; (4-{5-[bis (2-chloroethyl) amino]-1-methyl-2-benzimidazolyl} butyric acid; or 1H-benzimidazole-2-butanoic acid, 5-[bis(2-chloroethyl)amino]-1-methyl. Bendamustine is a compound of Formula (I):

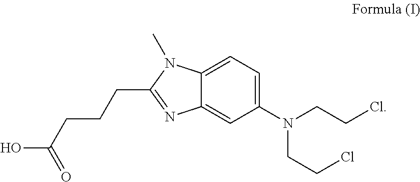 [SMILES: Cn1c(CCCC(=O)O)nc2cc(N(CCCl)CCCl)ccc21]

Bendamustine, polymorphs, or salts thereof are commercially available from a variety of sources.

As described herein, pharmaceutical formulations of bendamustine can comprise bendamustine or any of its pharmaceutically acceptable salts, isomers, racemates, enantiomers, hydrates, solvates, metabolites, polymorphs, or mixtures thereof suitable for pharmaceutical use. For example, bendamustine can be prepared in a variety of different forms, for example, chemical derivatives, solvates, hydrates, co-crystals, or salts. As used herein, “bendamustine” can include bendamustine or any of its pharmaceutically acceptable salts, isomers, racemates, enantiomers, hydrates, solvates, metabolites, polymorphs, or mixtures thereof.

Pharmaceutically acceptable salts are well known (see e.g., Stahl and Wermuth 2011 Pharmaceutical Salts: Properties, Selection & Use, Wiley-VCH, ISBN-10 3906390519). Except as otherwise noted herein, therefore, the process of the present disclosure can be carried out in accordance with such processes. Examples of pharmaceutically acceptable salts or acids include, but are not limited to: 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; hydrobromic acid; hydrochloric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; nitric acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); or undecylenic acid.

Bendamustine can be present in various compositions described herein, such as a pre-lyophilization formulation. For example, bendamustine can be present in a composition at a concentration of about 1 mg/ml to about 100 mg/ml. As another example, bendamustine can be present in a composition at a concentration of about 1 to 50 mg/ml. As another example, bendamustine can be present in a composition at a concentration of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, 61 mg/ml, about 62 mg/ml, about 63 mg/ml, about 64 mg/ml, about 65 mg/ml, about 66 mg/ml, about 67 mg/ml, about 68 mg/ml, about 69 mg/ml, about 70 mg/ml, 71 mg/ml, about 72 mg/ml, about 73 mg/ml, about 74 mg/ml, about 75 mg/ml, about 76 mg/ml, about 77 mg/ml, about 78 mg/ml, about 79 mg/ml, about 80 mg/ml, 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, 91 mg/ml, about 92 mg/ml, about 93 mg/ml, about 94 mg/ml, about 95 mg/ml, about 96 mg/ml, about 97 mg/ml, about 98 mg/ml, about 99 mg/ml, or about 100 mg/ml, or more. It is understood that recitation of the above discrete values includes a range between each recited value. Bendamustine-containing compositions described above can be used as a pre-lyophilization formulation.

In various embodiments, an anhydrous or a monohydrate state of Bendamustine can be used in a composition, such as a pre-lyophilization formulation.

In various embodiments, one or more polymorphs of Bendamustine can be used in a composition, such as a pre-lyophilization formulation. For examples of bendamustine polymorphs and preparation, crystallization, or characterization thereof, reference is made to the U.S. Pat. No. 8,445,524, which is incorporated herein by reference in its entirety. Except as otherwise noted herein, therefore, compositions and methods described herein can be in accordance with such knowledge of Bendamustine polymorphs.

Bendamustine can be prepared in different solid forms, in that they can be amorphous, can exist as different crystalline polymorphs, or in different solvation or hydration states (e.g., anhydrous or monohydrate). By varying the form of bendamustine, one can vary a physical property thereof. For example, solid forms of bendamustine can have different solubilities. Solid forms of bendamustine can differ in properties such as shelf-life, bioavailability, morphology, vapor pressure, density, color, or compressibility. Accordingly, variation of the solid state of bendamustine can modulate a physical or pharmacological property thereof.

Bendamustine can include one of more of the following four polymorphs: Form 1, Form 2, Form 3, or Form 4 (see generally, U.S. Pat. No. 8,445,524). These polymorphic solid forms can be identified, for example, by X-ray powder diffraction and characterized by one, two, three, four, five, or more reflection peaks that are characteristic of each polymorphic form. Crystalline polymorphs and amorphous bendamustine hydrochloride can also be identified by reference to their DSC thermograms, TGA thermograms, or GVS traces. Any of the solid forms of bendamustine described herein can be a component of a composition comprising bendamustine.

A composition described herein (e.g., a pre-lyophilization formulation) can include a Form 1 polymorph of bendamustine. Form 1 of bendamustine can be characterized as a white powder consisting of lath shaped particles. Form 1 of bendamustine can convert to a hydrate of bendamustine hydrochloride (Form 2) during 2 months of storage at 25° C./94% RH. A composition can include Form 1 of bendamustine at a concentration described herein. A Form 1 polymorph of bendamustine can be in an anhydrous or a monohydrate hydration state. A composition including Form 1 of bendamustine can be substantially free of other forms.

XRPD Data for Bendamustine HCl Form 1

| Angle | d value | Intensity | Intensity |
|---|---|---|---|
| (2-Theta) | (Angstrom) | (Counts) | (%) |
| 8.349 | 10.59033 | 110 | 6.8 |
| 13.503 | 6.55757 | 129 | 8 |
| 14.049 | 6.30377 | 394 | 24.5 |
| 16.824 | 5.26978 | 190 | 11.8 |
| 17.51 | 5.06473 | 172 | 10.7 |
| 18.452 | 4.80825 | 167 | 10.4 |
| 20.239 | 4.38767 | 130 | 8.1 |
| 20.904 | 4.24957 | 257 | 16 |
| 21.544 | 4.12484 | 295 | 18.3 |
| 21.972 | 4.04537 | 980 | 60.9 |
| 22.354 | 3.97705 | 210 | 13.1 |
| 22.922 | 3.87977 | 1213 | 75.4 |
| 23.305 | 3.81696 | 215 | 13.4 |
| 23.672 | 3.7586 | 317 | 19.7 |
| 24.851 | 3.58278 | 833 | 51.8 |
| 25.122 | 3.54475 | 1608 | 100 |
| 25.858 | 3.44558 | 173 | 10.7 |
| 26.35 | 3.38229 | 254 | 15.8 |
| 27.082 | 3.29256 | 437 | 27.2 |
| 27.591 | 3.23295 | 343 | 21.3 |
| 28.327 | 3.15055 | 704 | 43.8 |
| 29.155 | 3.06303 | 144 | 8.9 |
| 29.356 | 3.04246 | 151 | 9.4 |

A composition described herein (e.g., a pre-lyophilization formulation) can include a Form 2 polymorph of bendamustine. Form 2 of bendamustine, a monohydrate, can be characterized as a white powder consisting of rod shaped particles. A composition can include Form 2 of bendamustine at a concentration described herein. A Form 2 polymorph of bendamustine can be in an anhydrous or a monohydrate hydration state. A composition including Form 2 of bendamustine can be substantially free of other forms.

In some embodiments, a composition including a Form 2 polymorph of bendamustine and DMSO has increased stability or results in a lyophilized bendamustine composition with a shorter reconstitution time as compared to a composition with another polymorph.

XRPD Data for Bendamustine HCl Form 2

| Angle | d value | Intensity | Intensity |
|---|---|---|---|
| (2-Theta) | (Angstrom) | (Counts) | (%) |
| 10.169 | 8.69836 | 167 | 8.5 |
| 10.638 | 8.31653 | 1274 | 64.6 |
| 11.443 | 7.73271 | 155 | 7.9 |
| 12.46 | 7.10378 | 162 | 8.2 |
| 13.662 | 6.48137 | 186 | 9.4 |
| 15.055 | 5.88491 | 234 | 11.9 |
| 18.828 | 4.71319 | 631 | 32 |
| 19.724 | 4.50101 | 206 | 10.5 |
| 20.115 | 4.41437 | 955 | 48.4 |
| 20.451 | 4.34275 | 1017 | 51.6 |
| 20.95 | 4.24033 | 654 | 33.2 |
| 21.45 | 4.14261 | 371 | 18.8 |
| 22.15 | 4.01325 | 301 | 15.3 |
| 23.105 | 3.84943 | 1972 | 100 |
| 23.449 | 3.79375 | 373 | 18.9 |
| 23.859 | 3.72952 | 236 | 12 |
| 24.101 | 3.6926 | 271 | 13.7 |
| 24.511 | 3.6317 | 317 | 16.1 |
| 24.849 | 3.58309 | 290 | 14.7 |
| 25.204 | 3.53342 | 434 | 22 |
| 25.498 | 3.49344 | 320 | 16.2 |
| 25.843 | 3.44749 | 257 | 13 |
| 26.538 | 3.35877 | 788 | 40 |
| 27.248 | 3.27289 | 382 | 19.4 |
| 27.695 | 3.22103 | 402 | 20.4 |
| 28.018 | 3.18459 | 243 | 12.3 |
| 28.256 | 3.15834 | 248 | 12.6 |
| 28.487 | 3.13331 | 297 | 15 |
| 29.046 | 3.07423 | 352 | 17.9 |
| 29.255 | 3.0527 | 244 | 12.4 |

A composition described herein (e.g., a pre-lyophilization formulation) can include a Form 3 polymorph of bendamustine. Form 3 of bendamustine can be characterized as a white powder that was partially crystalline by XRPD. A composition can include Form 3 of bendamustine at a concentration described herein. A Form 3 polymorph of bendamustine can be in an anhydrous or a monohydrate hydration state. A composition including Form 3 of bendamustine can be substantially free of other forms.

XRPD Data for Bendamustine HCl Form 3

| Angle | d value | Intensity | Intensity |
|---|---|---|---|
| (2-Theta) | (Angstrom) | (Counts) | (%) |
| 3.85 | 22.95248 | 13.6 | 2.1 |
| 5.384 | 16.41406 | 16.3 | 2.5 |
| 5.75 | 15.37009 | 12.1 | 1.9 |
| 7.892 | 11.20261 | 40.4 | 6.2 |
| 10.575 | 8.36538 | 177 | 27.2 |
| 13.426 | 6.59478 | 30.1 | 4.6 |
| 13.636 | 6.49389 | 10.9 | 1.7 |
| 13.993 | 6.32893 | 36.3 | 5.6 |
| 14.7 | 6.0261 | 7.62 | 1.2 |
| 15.547 | 5.69958 | 121 | 18.6 |
| 15.734 | 5.63243 | 41.4 | 6.4 |
| 17.35 | 5.1112 | 25 | 3.8 |
| 17.608 | 5.0369 | 14.1 | 2.2 |
| 18.594 | 4.77186 | 55.1 | 8.5 |
| 18.85 | 4.70772 | 85.8 | 13.2 |
| 19.428 | 4.56899 | 80.2 | 12.3 |
| 19.749 | 4.49541 | 436 | 67 |
| 19.995 | 4.44068 | 173 | 26.6 |
| 21.3 | 4.17144 | 216 | 33.3 |
| 22.11 | 4.02037 | 233 | 35.8 |
| 23.328 | 3.81319 | 409 | 63 |
| 25.449 | 3.49996 | 393 | 60.5 |
| 25.571 | 3.48361 | 355 | 54.6 |
| 25.733 | 3.46204 | 294 | 45.3 |
| 26.083 | 3.41636 | 650 | 100 |
| 26.394 | 3.37675 | 305 | 46.9 |
| 26.61 | 3.34983 | 279 | 43 |
| 27.852 | 3.2032 | 393 | 60.5 |
| 27.977 | 3.1892 | 403 | 62 |
| 28.109 | 3.17455 | 392 | 60.3 |
| 29.039 | 3.07492 | 195 | 30 |

A composition described herein (e.g., a pre-lyophilization formulation) can include a Form 4 polymorph of bendamustine. Form 4 of bendamustine can be characterized as a white powder that was crystalline by XRPD. A composition can include Form 4 of bendamustine at a concentration described herein. A Form 4 polymorph of bendamustine can be in an anhydrous or a monohydrate hydration state. A composition including Form 4 of bendamustine can be substantially free of other forms.

XRPD Data for Bendamustine HCl Form 4

| Angle | d value | Intensity | Intensity |
|---|---|---|---|
| (2-Theta) | (Angstrom) | (Counts) | (%) |
| 3.86 | 22.88824 | 63.2 | 4.6 |
| 7.794 | 11.34336 | 120 | 8.8 |
| 10.267 | 8.61623 | 293 | 21.4 |
| 10.831 | 8.16867 | 1297 | 95 |
| 11.624 | 7.61314 | 149 | 10.9 |
| 11.804 | 7.4972 | 134 | 9.8 |
| 12.806 | 6.91286 | 169 | 12.4 |
| 14.077 | 6.29121 | 209 | 15.3 |
| 15.521 | 5.70899 | 376 | 27.5 |
| 16.038 | 5.5262 | 135 | 9.9 |
| 18.748 | 4.73313 | 168 | 12.3 |
| 19.636 | 4.52097 | 455 | 33.3 |
| 20.447 | 4.34345 | 1021 | 74.7 |
| 20.734 | 4.28411 | 793 | 58.1 |
| 21.227 | 4.18563 | 557 | 40.8 |
| 21.865 | 4.06498 | 202 | 14.8 |
| 22.263 | 3.99311 | 198 | 14.5 |
| 23.1 | 3.85031 | 306 | 22.4 |
| 23.579 | 3.77323 | 1366 | 100 |
| 23.95 | 3.71555 | 513 | 37.5 |
| 24.39 | 3.64947 | 250 | 18.3 |
| 24.548 | 3.62633 | 237 | 17.3 |
| 25.477 | 3.49624 | 266 | 19.5 |
| 25.81 | 3.45184 | 659 | 48.3 |
| 26.559 | 3.35619 | 258 | 18.9 |
| 27.101 | 3.29025 | 363 | 26.6 |
| 27.627 | 3.22885 | 818 | 59.9 |
| 28.415 | 3.14102 | 364 | 26.6 |

As described herein, the term “substantially free,” with regard to compositions that contain a particular form of bendamustine while being “substantially free” of other forms of the compound, can mean that the recited form can be associated with less than 10%, less than 5%, less than 2%, less than 1%, or less than 0.5% of another form of bendamustine.

Excipient

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005). Such formulations will contain a therapeutically effective amount of a biologically active agent described herein, which can be in purified form, together with a suitable amount of carrier so as to provide the form for proper administration to the subject.

For examples of formulations of bendamustine, reference is made to U.S. Pat. No. 8,445,524, U.S. Pat. No. 8,436,190, and International Application No. PCT/US2012/022561 (Published as WO 2012/103226), which are incorporated herein by reference in their entireties.

The term “formulation” refers to preparing a drug, e.g., bendamustine, in a form suitable for administration to a subject, such as a human. Thus, a “formulation” can include pharmaceutically acceptable excipients, including diluents or carriers.

The term “pharmaceutically acceptable” as used herein describes substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

Pharmaceutical formulations can comprise bendamustine or any of its pharmaceutically acceptable salts, isomers, racemates, enantiomers, hydrates, solvates, metabolites, polymorphs, and mixtures thereof suitable for pharmaceutical use.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers (e.g., change in pH, change in temperature, enzymes, water, or other physiological conditions or molecules).

Agents or compositions described herein can also be used in combination with other therapeutic modalities, as described further below. Thus, in addition to the therapies described herein, one may also provide to the subject other therapies known to be efficacious for treatment of the disease, disorder, or condition.

A “stable” formulation or composition can refer to any composition of bendamustine having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

A pharmaceutically acceptable excipient can be included in a pre-lyophilized bendamustine solution or a lyophilized bendamustine composition of the present disclosure. The pharmaceutically acceptable excipient can be dissolved in a suitable solvent.

For example, pharmaceutically acceptable excipients useful for the present application can include, without limitation thereto sodium phosphate, potassium phosphate, citric acid, tartaric acid, gelatin, glycine, and carbohydrates such as lactose, sucrose, maltose, mannitol, glycerin, dextrose, dextran, trehalose, and hetastarch or any mixtures of two or more thereof. An excipient may also comprise a pharmaceutically acceptable antioxidant, such as, for example, ascorbic acid, acetylcysteine, cysteine, sodium hydrogen sulfite, butyl-hydroxylanisole, butyl-hydroxytoluene, or alpha-tocopherol acetate.

Other excipients that can be used, if desired, include antioxidants, such as, without limitation thereto, ascorbic acid, acetylcysteine, cysteine, sodium hydrogen sulfite, butylated hydroxyanisole, butylated hydroxytoluene, alpha-tocopherol acetate, and chelating agents.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art, such as in Remington: The Science and Practice of Pharmacy, 20^{th} ed.; Gennaro, A. R., Ed.; Lippincott Williams & Wilkins: Philadelphia, Pa., 2000. Except insofar as any conventional media or agent is incompatible with an active ingredient, its use in the therapeutic compositions is contemplated. Supplementary active ingredients can also be incorporated into the compositions.

For example, compositions as described herein can comprise a pharmaceutically acceptable excipient in concentrations of about 1 mg/ml to about 100 mg/ml. As another example, compositions as described herein can comprise a pharmaceutically acceptable excipient in concentrations of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, 61 mg/ml, about 62 mg/ml, about 63 mg/ml, about 64 mg/ml, about 65 mg/ml, about 66 mg/ml, about 67 mg/ml, about 68 mg/ml, about 69 mg/ml, about 70 mg/ml, 71 mg/ml, about 72 mg/ml, about 73 mg/ml, about 74 mg/ml, about 75 mg/ml, about 76 mg/ml, about 77 mg/ml, about 78 mg/ml, about 79 mg/ml, about 80 mg/ml, 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, 91 mg/ml, about 92 mg/ml, about 93 mg/ml, about 94 mg/ml, about 95 mg/ml, about 96 mg/ml, about 97 mg/ml, about 98 mg/ml, about 99 mg/ml, or about 100 mg/ml, or more. It is understood that recitation of the above discrete values includes a range between each recited value.

In some embodiments, a pre-lyophilized bendamustine solution or a lyophilized bendamustine composition includes mannitol. It has been reported that mannitol, used as a bulking agent for bendamustine formulation, cannot be dissolved in a completely organic solvent system and, thus, requires water be part of a solvent system for any bendamustine formulation (WO2012/103226, page 4). In contrast to these teachings, the present disclosure demonstrates that mannitol can in fact be used with 100% DMSO or other elevated levels of DMSO. This is a surprising result in light of prior reports.

For example, a composition described herein can include mannitol at a concentration of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, 61 mg/ml, about 62 mg/ml, about 63 mg/ml, about 64 mg/ml, about 65 mg/ml, about 66 mg/ml, about 67 mg/ml, about 68 mg/ml, about 69 mg/ml, about 70 mg/ml, 71 mg/ml, about 72 mg/ml, about 73 mg/ml, about 74 mg/ml, about 75 mg/ml, about 76 mg/ml, about 77 mg/ml, about 78 mg/ml, about 79 mg/ml, about 80 mg/ml, 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, 91 mg/ml, about 92 mg/ml, about 93 mg/ml, about 94 mg/ml, about 95 mg/ml, about 96 mg/ml, about 97 mg/ml, about 98 mg/ml, about 99 mg/ml, or about 100 mg/ml, or more. It is understood that recitation of the above discrete values includes a range between each recited value.

Pre-Lyophilization Bendamustine Solution

As described herein, bendamustine can be compounded or formulated with an organic solvent, such as DMSO, to form a stable pre-lyophilized dispersion (e.g., a solution). A dispersion is understood to be a system composed in which particles are dispersed in a continuous phase of a different composition, or in other words a dispersed substance and a medium in which it is dispersed. Dispersions can include a coarse dispersion (i.e., a suspension), a colloid, or a solution. A solution is understood to be homogeneous or semi-homogeneous mixture formed by a solid, liquid, or gaseous substance being mixed with a liquid. The balance of the discussion herein employs “solution” but one of ordinary skill will understand such discussion applies equally to a “dispersion” generally.

A pre-lyophilized bendamustine solution can be aseptically filtered into a sterile container, filled into an appropriate sized vial, or loaded into a vacuum dryer.

A pre-lyophilized solution including bendamustine and an organic solvent (e.g., DMSO) can be stable for at least about 1 hour to at least about 1 week, when stored at, e.g., about 2° C. to about 25° C., and can be readily available for lyophilization. For example, a pre-lyophilized solution including bendamustine and an organic solvent (e.g., DMSO) can be stable for at least about 1 hour to at least about 1 day, at least about 2 days, at least about 3 days, at least about 4 days, at least about 5 days, or at least about 6 days, when stored at, e.g., about 2° C. to about 25° C., and can be readily available for lyophilization.

For example, a pre-lyophilized solution including bendamustine and an organic solvent (e.g., DMSO) can be stable for at least about 1 hour, at least about 5 hours, at least about 10 hours, at least about 15 hours, at least about 17 hours, at least about 20 hours, at least about 25 hours, at least about 30 hours, at least about 35 hours, at least about 40 hours, at least about 45 hours, at least about 50 hours, at least about 55 hours, at least about 60 hours, at least about 65 hours, at least about 70 hours, or at least about 72 hours, or more, when stored at, e.g., about 2° C. to about 25° C., and can be readily available for lyophilization. It is understood that recitation of the above discrete values includes a range between each recited value.

As another example, the pre-lyophilized solution including bendamustine and an organic solvent (e.g., DMSO) can be stable for at least about 1 day, at least about 2 days, at least about 3 days, at least about 4 days, at least about 5 days, at least about 6 days, at least about 7 days, at least about 8 days, at least about 9 days, or at least about 10 days, or more. It is understood that recitation of the above discrete values includes a range between each recited value.

A pre-lyophilized solution including bendamustine and an organic solvent (e.g., DMSO) can be formulated by: (1) dissolving an excipient, such as mannitol, in an organic solvent (e.g., about 35-100% organic solvent v/v to water) at, e.g., ambient temperature; (2) adding bendamustine (e.g., bendamustine hydrochloride) to obtain a desired concentration; and (3) adding an organic solvent (e.g., about 35-100% organic solvent v/v to water) to achieve a final volume. Such pre-lyophilized solution can be readily available for lyophilization, e.g., step (4) subjecting the bulk solution to lyophilization.

Although the preceding steps are shown in a certain order, it is understood that one skilled in the art can change the order of the steps and quantities as desired.

A pre-lyophilized solution can be sterilized prior to vacuum drying, sterilization sometimes being performed by aseptic filtration, e.g., through a 0.22 μm or smaller pore filter. Multiple sterilization filters or procedures can be used. Sterilization of a solution or dispersion can be achieved using other methods known in the art, e.g., by exposure to radiation.

After filtration, a solution can be subjected to vacuum drying. Generally, the filtered solution can be introduced into a receiving vessel (e.g., sterile receiving vessel), and then transferred to a suitable container or containers in which the formulation can be effectively dried under vacuum. The formulation can be effectively and efficiently dried in a container in which the product is to be marketed, such as, without limitation, a vial, as described herein and as known in the art.

As described herein, a pre-lyophilization bendamustine solution can contain small amounts or trace amounts of “bendamustine-related” impurities that can be the result of decomposition or degradation of bendamustine during pharmaceutical product manufacturing or storage, or can be artifacts from processes for synthesizing the drug.

Lyophilization

As described herein, a stable formulation of bendamustine with an organic solvent, such as DMSO, can be lyophilized to produce a lyophilized bendamustine composition having increased stability, increased bulk concentration, lower total impurities, or decreased reconstitution time. Lyophilization processes are well known; see e.g., Rey et al., Freeze-Drying/Lyophilization of Pharmaceutical and Biological Products, Drugs and Pharmaceutical Sciences, 2004, Marcel Dekker, Inc., New York, N.Y. Except as otherwise noted herein, therefore, the compositions and methods of the present disclosure can be in accordance with such processes.

The lyophilized bendamustine formulations as described herein were found to have significantly lower reconstitution times than the time listed in the reference listed drug (RLD), Treanda®, package insert (“the lyophilized powder should completely dissolve in 5 minutes”).

The lyophilized bendamustine formulations as described herein were found to have comparable or better impurity levels and chromatographic profile as Treanda®, the reference listed drug (RLD) for bendamustine HCl for injection.

The lyophilized bendamustine formulations as described herein were found to exhibit less impurities than Treanda®, even with increased hold-time. Maximum hold time for a bulk solution can be important to establish during product development, as it can define process controls used during product manufacture. For example, temperature-sensitive bulk solutions generally must be maintained at low temperatures (i.e., 2-8° C.) whenever possible (pre-filtration, post-filtration/pre-filling or post-filling) to prevent degradation. But in certain cases, as described herein, a bulk solution can be optimized to allow for certain processing conditions. In other words, a bulk solution including bendamustine and DMSO can be conveniently maintained at a temperature higher than 2-8° C. (e.g., room temperature).

The term “lyophilization” can refer to processes, in which bendamustine, together with any desired pharmaceutical excipients, is dissolved in a solvent and then subjected to a procedure that involves placing into a dryer and establishing a low shelf temperature, e.g., from −50° C. to 25° C., applying vacuum to obtain a powder residue, and subsequently drying under reduced vacuum to remove residual solvent. Lyophilization processing can be suitable for injectables because it can be conducted under sterile conditions, which can be a requirement for parenteral dosage forms.

Lyophilization, freeze-drying, or vacuum drying, as described herein can be processes in which solvent is removed from a solution or dispersion after it is frozen and placed under a vacuum, allowing the solvent to change directly from a solid to a vapor without passing through a liquid phase. A lyophilization process can include various phases: a freezing phase; a primary drying phase (sublimation); or a secondary drying phase (desorption). Such phases can be separate, unique, independent, or interdependent. A lyophilization process can be optimized, as described herein, to enhance product stability as well as decrease the manufacturing costs.

Advantages of lyophilization can include ease of processing a liquid, which simplifies aseptic handling; enhanced stability of a dry powder; removal of a solvent without excessive heating of a product; enhanced product stability in a dry state; or rapid or easy dissolution of reconstituted product. With a lyophilization process, a product can be dried without having to use elevated temperatures, thereby eliminating adverse thermal effects, and can be stored in a dry state, for which there can be decreased stability problems. Freeze dried products can be more soluble, dispersions can be stabilized, or products subject to degradation by oxidation or hydrolysis can be protected.

As described herein, a lyophilized formulation of bendamustine can be achieved following removal of an organic solvent and water from a pre-lyophilization composition. An organic solvent used to prepare a pre-lyophilization composition can be as described above. An organic solvent can be used individually or in any combinations of two or more. A desirable organic solvent can form a stable solution with bendamustine and not appreciably degrade or deactivate the drug. Solubility of bendamustine in a selected solvent can be high enough to form commercially useful concentrations of the drug. An organic solvent can be capable of being removed easily from an aqueous dispersion or solution of the drug product, e.g., through lyophilization.

Lyophilized Bendamustine Composition

A pre-lyophilization bendamustine solution can be dried to obtain a lyophilized bendamustine composition. A lyophilized bendamustine composition of the present disclosure includes bendamustine. In addition to bendamustine, a lyophilized bendamustine composition can include an excipient, an organic solvent (e.g., a trace amount of an organic solvent left after lyophilization), water, or impurities. As shown herein, a lyophilized bendamustine composition prepared as described herein can have a decreased amount of impurities, an increased amount bendamustine, a decreased amount of water, or a decreased amount of impurities, as compared to a lyophilized bendamustine composition prepared according to conventional methods.

A lyophilized bendamustine composition can be stored as a stable dried powder. A lyophilized bendamustine composition can be stored as a stable dried powder in a closed container.

A lyophilized bendamustine composition can be stored as a stable dried powder up to about 60° C. For example, a lyophilized bendamustine composition can be stored as a stable dried powder at about 0° C. to about 60° C. As another example, a lyophilized bendamustine composition can be stored as a stable dried powder at about 5° C. to about 25° C.

A lyophilized bendamustine composition can be stored as a stable dried powder for at least about 3 years or more. For example, a lyophilized bendamustine composition can be stored as a stable dried powder for about six months to about 3 years or more (e.g., 3 years or more).

As another example, a lyophilized composition including bendamustine can be stable for at least about 3 years, at least about 2 years, at least about 1.5 years, at least about 1 year, at least about 11 months, at least about 10 months, at least about 9 months, at least about 8 months, at least about 7 months, at least about 6 months, at least about 5 months, at least about 4 months, at least about 3 months, at least about 2 months, at least about 1 month, at least about 3 weeks, at least about 2 weeks, at least about 1 week, at least about 6 days, at least about 5 days, at least about 4 days, at least about 2 days, or at least about 1 day when stored at an acceptable temperature as described herein (e.g., up to about 60° C.; from about 0° C. to about 60° C.; or from about 5° C. to about 25° C.).

Bendamustine and Excipient.

A lyophilized bendamustine composition can be stored (e.g., in a vial) in an amount of bendamustine from about 1 mg to about 100 mg and an amount of an excipient (e.g., mannitol) from about 5 mg to about 2 g. For example, a storage amount of a lyophilized bendamustine composition can include about 10 mg to about 500 mg of bendamustine and about 5 mg to about 2 g of an excipient (e.g., mannitol). As another example, a storage amount of a lyophilized bendamustine composition can include about 25 mg to about 100 mg of bendamustine and about 10 mg to about 300 mg of an excipient (e.g., mannitol). One of ordinary skill in the art will recognize the above numbers are relative and that a lyophilized bendamustine composition can be stored in any convenient amount.

A lyophilized bendamustine composition, although typically in vial packaging, may be stored in any suitable container (e.g., a use container), such as ampoules, syringes, or co-vials, which are capable of maintaining a sterile environment. A “vial” can include any walled container, whether rigid or flexible. Such a container can be made of glass or plastic, provided that the material does not interact with the bendamustine formulation. A container closure can be a stopper, such as a sterile rubber stopper (e.g., a bromobutyl rubber stoppers), which can afford a hermetic seal.

Water Content.

In addition to bendamustine, a lyophilized bendamustine composition of the present disclosure can include water. A lyophilized bendamustine composition can have a water content in the range of about 0% to about 20% by weight. For example, a lyophilized bendamustine composition can have a water content in the range of about 0%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% by weight. It is understood that recitation of the above discrete values includes a range between each recited value. As another example, the pre-lyophilization process can control the water content in the lyophilized final product such that the water content can be about 0%, less than about 0.1%, less than about 0.2%, less than about 0.3%, less than about 0.4%, less than about 0.5%, less than about 0.6%, less than about 0.7%, less than about 0.8%, less than about 0.9%, less than about 1.0%, less than about 1.1%, less than about 1.2%, less than about 1.3%, less than about 1.4%, less than about 1.5%, less than about 1.6%, less than about 1.7%, less than about 1.8%, less than about 1.9%, or less than about 2.0%. It is understood that recitation of the above discrete values includes a range between each recited value.

Impurities.

In addition to bendamustine, a lyophilized bendamustine composition of the present disclosure can include impurities. As described herein, a pre-lyophilized solution or lyophilized bendamustine compositions can comprise drug-related impurities within commercially acceptable limits, the impurity concentrations being maintained during storage for commercially relevant times.

For examples of identification and characterization of various impurities of bendamustine, reference is made to U.S. Pat. No. 8,445,524, U.S. Pat. No. 8,436,190, and International Application No. PCT/US2012/022561 (Published as WO 2012/103226), which are incorporated herein by reference in their entireties.

A lyophilized bendamustine composition can include a residual amount of an organic solvent. For example, a lyophilized bendamustine composition can include a trace amount of an organic solvent. As used herein, a “trace amount” of an organic solvent can mean an amount of solvent that can be equal to or below recommended levels for pharmaceutical products, for example, as recommended by ICH guidelines (International Conferences on Harmonization, Impurities-Guidelines for Residual Solvents. Q3C, published in the U.S. Federal Register, Vol. 62, No. 247, pages 67377-67388, 1997).

As described herein, a lyophilized bendamustine composition can contain small amounts or trace amounts of “bendamustine-related” impurities that can be the result of decomposition or degradation of bendamustine during pharmaceutical product manufacturing or storage, or can be artifacts from processes for synthesizing the drug.

A lyophilized bendamustine composition can include bendamustine-related impurities totaling less than about 8.0%. As another example, a lyophilized bendamustine composition can include bendamustine-related impurities totaling less than about 7.0%, less than about 6.5%, less than about 6.0%, less than about 5.5%, less than about 5.0%, less than about 4.5%, less than about 4.0%, less than about 3.5%, less than about 3.0%, less than about 2.5%, less than about 2.0%, less than about 1.5%, less than about 1%, less than about 0.9%, less than about 0.8%, less than about 0.75%, less than about 0.7%, less than about 0.65%, less than about 0.6%, less than about 0.55%, less than about 0.5%, less than about 0.45%, less than about 0.4%, less than about 0.35%, less than about 0.3%, less than about 0.25%, less than about 0.2%, less than about 0.15%, or less than about 0.1%. It is understood that recitation of the above discrete values includes a range between each recited value.

As another example, a lyophilized bendamustine composition can include bendamustine-related impurities totaling about 0% to less than about 8.0%. As another example, a lyophilized bendamustine composition can include bendamustine-related impurities totaling about 0% to less than about 7.5%, less than about 7.0%, about 0% to less than about 6.5%, about 0% to less than about 6.0%, about 0% to less than about 5.5%, about 0% to less than about 5.0%, about 0% to less than about 4.5%, about 0% to less than about 4.0%, about 0% to less than about 3.5%, about 0% to less than about 3.0%, about 0% to less than about 2.5%, about 0% to less than about 2.0%, about 0% to less than about 1.5%, about 0% to less than about 1%, about 0% to less than about 0.9%, about 0% to less than about 0.8%, about 0% to less than about 0.75%, about 0% to less than about 0.7%, about 0% to less than about 0.65%, about 0% to less than about 0.6%, about 0% to less than about 0.55%, about 0% to less than about 0.5%, about 0% to less than about 0.45%, about 0% to less than about 0.4%, about 0% to less than about 0.35%, about 0% to less than about 0.3%, about 0% to less than about 0.25%, about 0% to less than about 0.2%, about 0% to less than about 0.15%, or about 0% to less than about 0.1%. It is understood that recitation of the above discrete values includes a range between each recited value.

As another example, a lyophilized bendamustine composition can include bendamustine-related impurities totaling about 0%, about 0.10%, about 0.11%, about 0.12%, about 0.13%, about 0.14%, about 0.15%, about 0.16%, about 0.17%, about 0.18%, about 0.19%, about 0.20%, about 0.21%, about 0.22%, about 0.23%, about 0.24%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1%, about 1.5%, about 2%, about 2.5%, about 3%, about 3.5%, about 4%, about 4.5%, about 5%, about 5.5%, about 6%, about 6.5%, about 7%, about 7.5%, or about 8%. It is understood that recitation of the above discrete values includes a range between each recited value.

Reconstitution

As described herein, a lyophilized bendamustine composition prepared according to methods of the present disclosure can provide for decreased reconstitution times as compared to lyophilized bendamustine compositions prepared by conventional methods.

A lyophilized bendamustine composition can be reconstituted with an aqueous solution (e.g., sterile water, saline, or a mixture thereof) to provide a liquid formulation of bendamustine. A reconstituted bendamustine solution can be suitable for administration (e.g., injection, parenteral injection, intravenous administration). A reconstituted bendamustine solution can include a pharmaceutically acceptable excipient or adjuvant, as described herein. For example, a lyophilized bendamustine composition can be reconstituted with about 5% dextrose injection, lactated Ringer's and dextrose injection, about 0.9% sodium chloride, about 5% dextrose in water (D5W), Lactated Ringers solution, or about 0.45% sodium chloride/about 2.5% dextrose, or sterile water for injection, or a variant thereof.

In some embodiments, reconstitution time of a lyophilized bendamustine composition described herein can be less than about 300 seconds. For example, reconstitution time of a lyophilized bendamustine composition described herein can be less than about 120 seconds. As another example, reconstitution time of a lyophilized bendamustine described herein can be less than about 300 seconds, less than about 290 seconds, less than about 280 seconds, less than about 270 seconds, less than about 260 seconds, less than about 250 seconds, less than about 240 seconds, less than about 230 seconds, less than about 220 seconds, less than about 210 seconds, less than about 200 seconds, less than about seconds, less than about 190 seconds, less than about 180 seconds, less than about 170 seconds, less than about 160 seconds, less than about 150 seconds, less than about 140 seconds, less than about 130 seconds, less than about 120 seconds, less than about 110 seconds, less than about 100 seconds, less than about 90 seconds, less than about 80 seconds, less than about 70 seconds, less than about 60 seconds, less than about 59 seconds, less than about 58 seconds, less than about 57 seconds, less than about 56 seconds, less than about 55 seconds, less than about 54 seconds, less than about 53 seconds, less than about 52 seconds, less than about 51 seconds, less than about 50 seconds, less than about 49 seconds, less than about 48 seconds, less than about 47 seconds, less than about 46 seconds, less than about 45 seconds, less than about 44 seconds, less than about 43 seconds, less than about 42 seconds, less than about 41 seconds, less than about 40 seconds, less than about 39 seconds, less than about 38 seconds, less than about 37 seconds, less than about 36 seconds, less than about 35 seconds, less than about 34 seconds, less than about 33 seconds, less than about 32 seconds, less than about 31 seconds, less than about 30 seconds, less than about 29 seconds, less than about 28 seconds, less than about seconds, less than about 27 seconds, less than about 26 seconds, less than about 25 seconds, less than about 24 seconds, less than about 23 seconds, less than about 22 seconds, less than about 21 seconds, less than about 20 seconds, less than about 19 seconds, less than about 18 seconds, less than about 17 seconds, less than about 16 seconds, less than about 15 seconds, less than about 14 seconds, less than about 13 seconds, less than about 12 seconds, less than about 11 seconds, less than about 10 seconds, less than about 9 seconds, less than about 8 seconds, less than about 7 seconds, less than about 6 seconds, less than about 5 seconds, less than about 4 seconds, less than about 3 seconds, less than about 2 seconds, or less than about 1 second. It is understood that recitation of the above discrete values includes a range between each recited value.

In some embodiments, reconstitution time of a lyophilized bendamustine composition described herein can be less than about 20 minutes. For example, reconstitution time can be less than about 19.5 minutes, less than about 19 minutes, less than about 18.5 minutes, less than about 18 minutes, less than about 17.5 minutes, less than about 17 minutes, less than about 16.5 minutes, less than about 16 minutes, less than about 15.5 minutes, less than about 15 minutes, less than about 14.5 minutes, less than about 14 minutes, less than about 13.5 minutes, less than about 13 minutes, less than about 12.5 minutes, less than about 12 minutes, less than about 11.5 minutes, less than about 11 minutes, less than about 10.5 minutes, less than about 10 minutes, less than about 9.5 minutes, less than about 9 minutes, less than about 8.5 minutes, less than about 8 minutes, less than about 7.5 minutes, less than about 7 minutes, less than about 6.5 minutes, less than about 6 minutes, less than about 5.5 minutes, less than about 5 minutes, less than about 4.5 minutes, less than about 4 minutes, less than about 3.5 minutes, less than about 3 minutes, less than about 2.5 minutes, less than about 2 minutes, less than about 1.5 minutes, less than about 1 minute, or less than about 0.5 minutes. It is understood that recitation of the above discrete values includes a range between each recited value.

As another example, reconstitution time can be about 1 second up to about 20 minutes, about 1 second to about 15 minutes, about 1 second to about 10 minutes, about 1 second to about 5 minutes, about 1 second to about 4 minutes, about 1 second to about 3 minutes, about 1 second to about 2 minutes, or about 1 second to about 1 minute.

As described herein, a reconstituted bendamustine solution can contain small amounts or trace amounts of “bendamustine-related” impurities that can be the result of decomposition or degradation of bendamustine during pharmaceutical product manufacturing or storage, or can be artifacts from processes for synthesizing the drug.

Administration

Agents and compositions described herein can be administered according to methods described herein in a variety of means known to the art. For examples of administration of bendamustine compositions, reference is made to U.S. Pat. No. 8,445,524, U.S. Pat. No. 8,436,190, and International Application No. PCT/US2012/022561 (Published as WO 2012/103226), which are incorporated herein by reference in their entireties.

Administration of a bendamustine composition can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems can: provide for intracellular delivery; tailor agent release rates; increase the proportion of agent that reaches its site of action; improve the transport of the agent to its site of action; allow co-localized deposition with other agents or excipients; improve the stability of the agent in vivo; prolong the residence time of the agent at its site of action by reducing clearance; decrease the nonspecific delivery of the agent to non-target tissues; decrease irritation caused by the agent; decrease toxicity due to high initial doses of the agent; alter the immunogenicity of the agent; decrease dosage frequency, improve taste of the product; or improve shelf life of the product.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to pre-lyophilized bendamustine solutions or lyophilized bendamustine compositions in vials or other suitable containers. Such packaging of the components separately can, if desired, be presented in a pack or dispenser device which may contain one or more unit dosage forms containing the composition. The pack can, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### General Formulation Protocol

The following example describes the general protocol for the formulation of bendamustine compositions in Examples 1-8.

Bendamustine HCl was an anhydrous form with a water content level of nmt 1.0%. The Bendamustine HCl used herein was not the Form 1 polymorph and was at least similar to an anhydrous version of the Form 2 polymorph. A sample of Bendamustine HCl anhydrous (with a thickness of about 2 mm was is saturated in water after 60 minutes and the water level reached about 4.7-4.8%. Exposing the Bendamustine HCl to atmospheric conditions for 1 hour caused the % water to increase to about 4.5%, corresponding to the monohydrate form (MW Bendamustine HCl=394.7 g/mol and H₂O=18 g/mol; [(394.7+18)/394.7]100=104.6%).

First, bendamustine was formulated in 100% DMSO and the resulting product was lyophilized. The residual DMSO in the product is analyzed for acceptable levels.

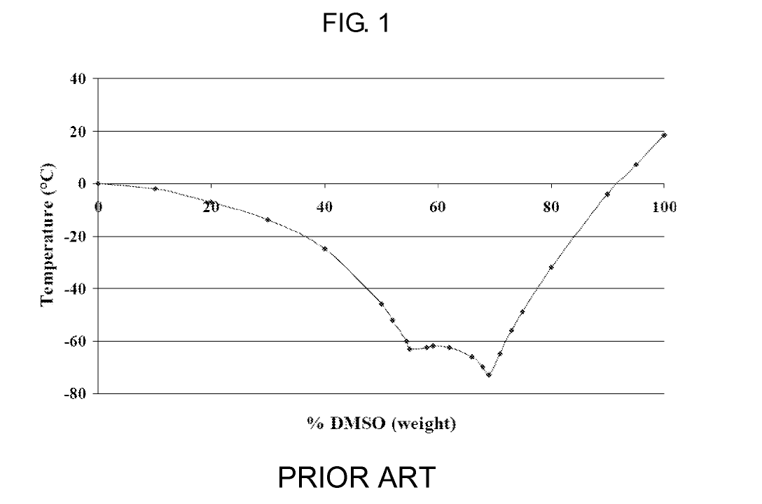

Second, bendamustine in DMSO/water mixture (see e.g., FIG. 1 for freezing point data) was formulated.

Third, a concentration of about 20 mg/ml at different temperatures was targeted to determine a formulation that is stable and has a bendamustine HCl concentration of not less than (NLT) 20 mg/ml.

Fourth, a 25 mg/ml bendamustine solution in DMSO or DMSO/water mixture was prepared. Mannitol was not soluble in 100% TBA. Different solvents like isopropyl alcohol and benzyl alcohol/water mixtures could also be used to produce bioequivalent products. Both DMSO and dimethylformamide (DMF) will boil below 50° C. if this vacuum is reduced from 760 torr to 5 torr (from 1 bar to 6.6 mbar) during lyophilization.

### Example 2

### Lyophilization

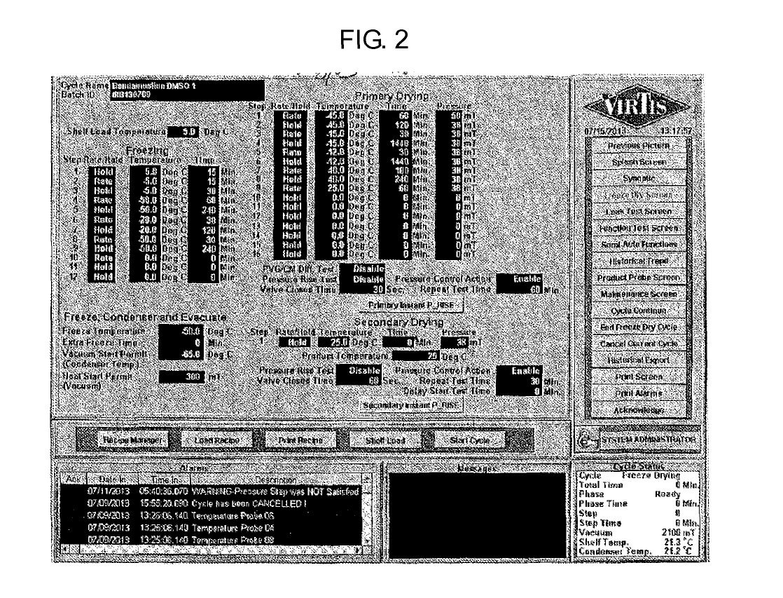

The following Example describes the parameters for lyophilization in Examples 1-8. The freeze dryer used in Examples 1-8 was a Virtis Model (Genesis 25 xL). Parameters used for freeze drying can be found in FIG. 2.

The pre-lyophilization formulation included bendamustine hydrochloride at a concentration of about 15-25 mg/ml, mannitol at a concentration of about 25.5-42.5 mg/ml, and dimethyl sulfoxide (DMSO) at a concentration of about 40-100% v/v in water. The pre-lyophilization formulations described herein are physically and chemically stable for periods of time that are suitable for pharmaceutical manufacturing and lyophilization.

Extensive lyophilization cycle development was conducted to produce lyophilized bendamustine samples that demonstrated acceptable physical properties and comparable chemical properties, relative to Treanda®, the reference listed drug product (RLD) for bendamustine HCl for injection. The cycles feature an annealing step to facilitate crystallization of mannitol, which accelerates DMSO (and water) removal during the primary drying phase. Likewise, use of DMSO allows for lower vacuum levels to be used during the primary drying phase without risk of powder ejection. The lyophilization cycle parameters are described in TABLE 1.

TABLE 1
A Lyophilization Cycle Protocol for Bendamustine
HCl for Injection (100 mg/vial)

| | | | | |
|---|---|---|---|---|
| | Rate or | Temp | Time | Vacuum |
| Step | Hold Step | (° C.) | (hr) | (mT) |

| | | | | | |
|---|---|---|---|---|---|
| 1. | Loading | Hold | 5 | 0.25 | — |
| 2. | Freezing | Rate | −5 | 0.25 | — |
| 3. | Freezing | Hold | −5 | 0.50 | — |
| 4. | Freezing | Rate | −50 | 1.00 | — |
| 5. | Freezing | Hold | −50 | 4.00 | — |
| 6. | Annealing | Rate | −20 | 0.50 | — |
| 7. | Annealing | Hold | −20 | 2.00 | — |
| 8. | Freezing | Rate | −50 | 0.50 | — |
| 9. | Freezing | Hold | −50 | 4.00 | — |
| 10. | Primary drying | Rate | −45 | 1.00 | 50 |
| 11. | Primary drying | Hold | −45 | 2.00 | 38 |
| 12. | Primary drying | Rate | −15 | 0.50 | 38 |
| 13. | Primary drying | Hold | −15 | 24.00 | 38 |
| 14. | Primary drying | Rate | −12 | 0.50 | 38 |
| 15. | Primary drying | Hold | −12 | 24.00 | 38 |
| 16. | Secondary drying | Rate | 40 | 3.00 | 38 |
| 17. | Secondary drying | Hold | 40 | 4.00 | 38 |
| 18. | Secondary drying | Rate | 25 | 1.00 | 38 |

| | | | | |
|---|---|---|---|---|
| Total Cycle Time (hr) | — | — | 73.00 | — |

Following lyophilization, the samples described herein contained about 100 mg/vial bendamustine HCl, about 170 mg/vial mannitol, and trace amounts of DMSO and water. The physical and chemical properties for a series of bendamustine formulations (before and after lyophilization) are summarized in the following examples.

### Example 3

### Impurity Analysis

The following Example describes the procedure and chromatogram results for the 070813 and 071513 impurity analysis sequences.

Mobile Phase A: 10 mM Potassium Hexafluorophosphate buffer, pH 3.0. Mobile Phase B: HPLC-grade Acetonitrile. Diluent: A 60:40 mixture of Mobile Phase A and Mobile Phase B. Impurity standard: Bendamustine HCl was exposed for 1 hour at room temperature under bench to achieve H₂O saturation. A solution of 2 μg/ml Bendamustine HCl in Diluent was prepared.

The liquid chromatography (LC) protocol is as follows.

Instrument: Dionex UltiMate 3000 equipped with a UV Detector and cooled Autosampler. Column: Shim-pack VP-ODS 250 L×4.6 mm, 5 μm. Flow Rate: about 0.8 ml/minute. Detector Wavelength: 233 nm, 8 nm bandwidth. Injection volume: 25 μl. Column Temperature: 25° C. Autosampler Tray Temperature: 5° C.

TABLE 2
Gradient

| Time | Mobile Phase B |
|---|---|
| [min] | (%) |
| 0.0 | 40.0 |
| 45.0 | 40.0 |
| 45.1 | 90.0 |
| 55.0 | 90.0 |
| 55.1 | 40.0 |
| 65.0 | 40.0 |

Samples for impurity analysis were prepared as follows.

6IB130708-1.

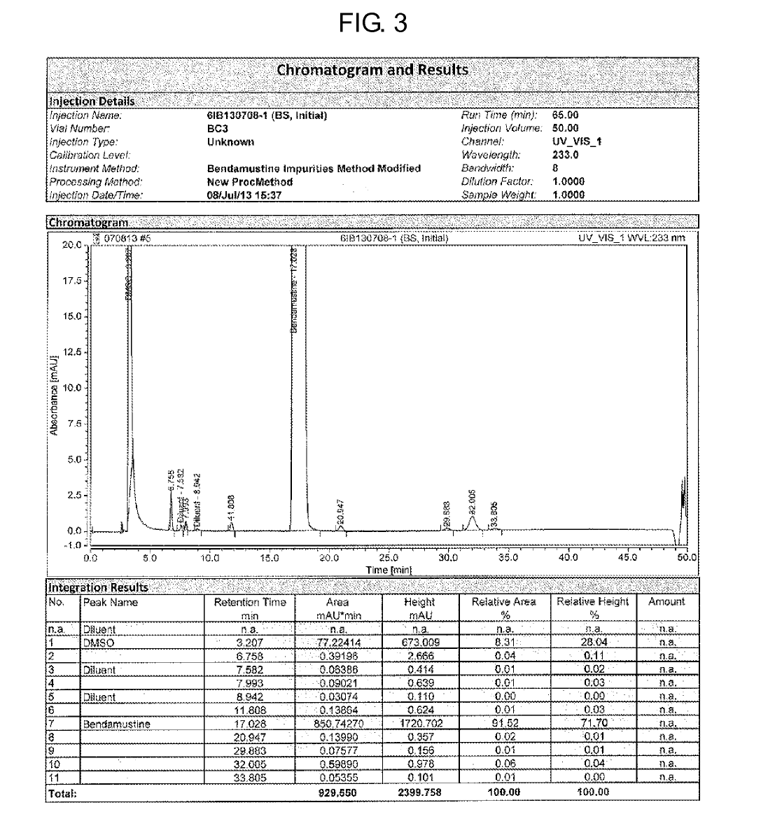

6IB130708-1 (bulk solution (BS)=25 mg/ml active pharmaceutical ingredient (API) in 100% DMSO) compounded at room temperature (see e.g., Example 4, TABLE 3, FIG. 3). A 200 μg/ml solution of Bendamustine HCl in Diluent was prepared.

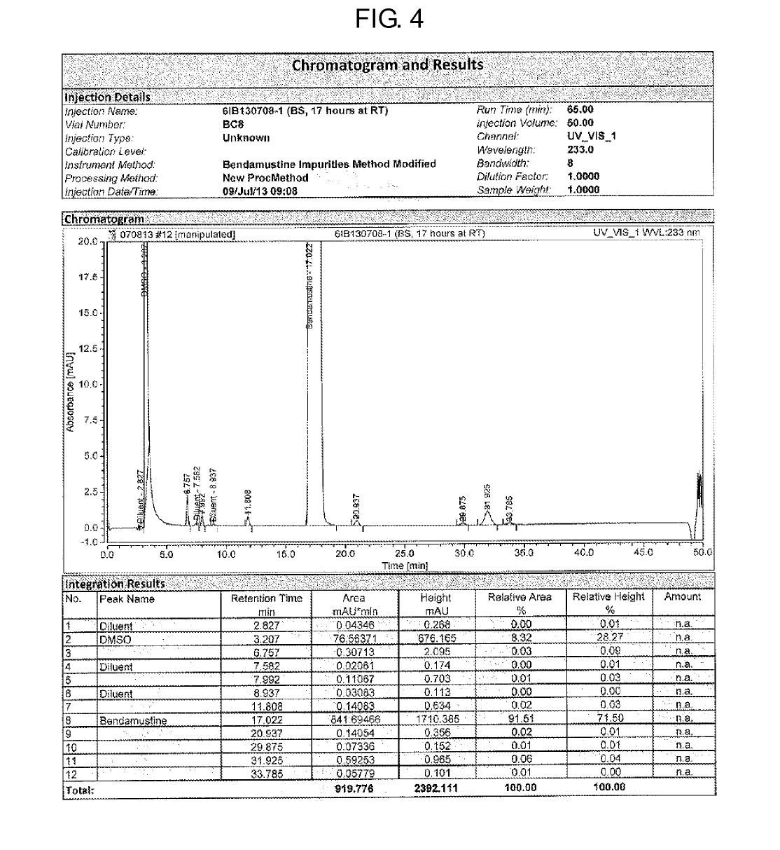

6IB130708-1/hold at room temperature (RT) for 17 hrs (see e.g., Example 4, TABLE 3, FIG. 4). A 200 μg/ml solution of Bendamustine HCl in Diluent was prepared.

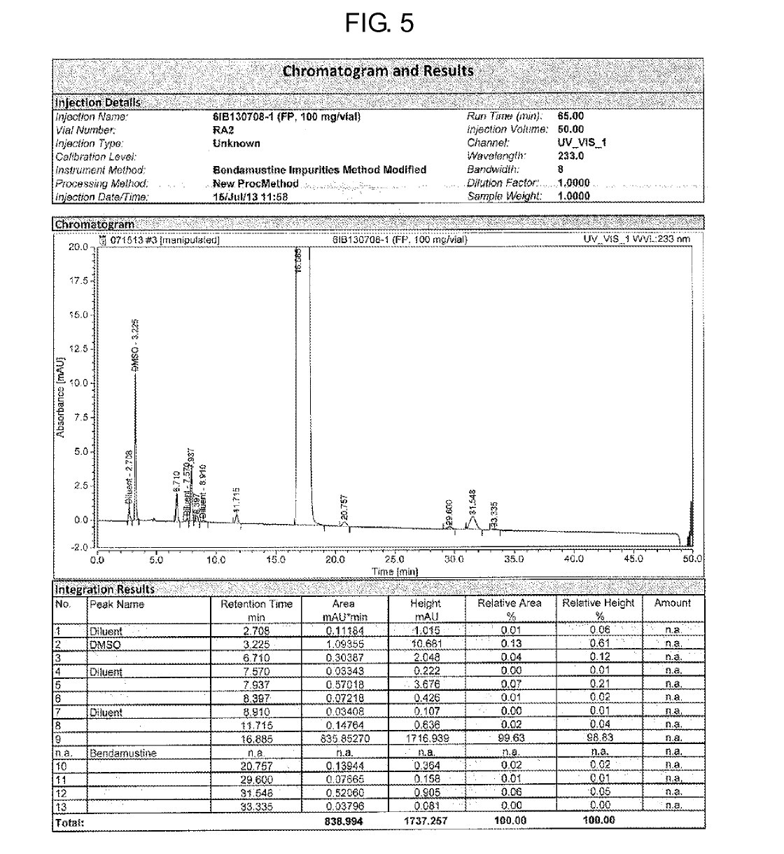

6IB130708-1 (lyophilized) 100 mg/vial (see e.g., Example 4, TABLE 3, FIG. 5). The final product was reconstituted with Diluent, and then a 200 μg/ml solution of Bendamustine HCl in Diluent was prepared.

6IB130708-2.

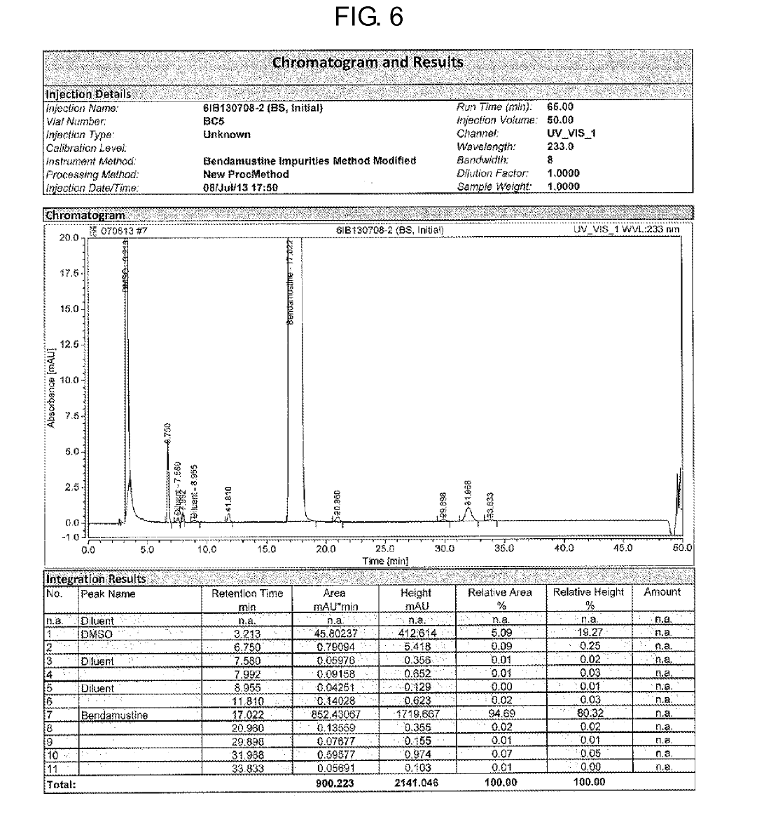

6IB130708-2 (bulk solution (BS)=25 mg/ml active pharmaceutical ingredient (API) in 50/50 DMSO/H₂O) compounded at 15° C. (see e.g., Example 5, TABLE 4, FIG. 6). A 200 μg/ml solution of Bendamustine HCl in Diluent was prepared.

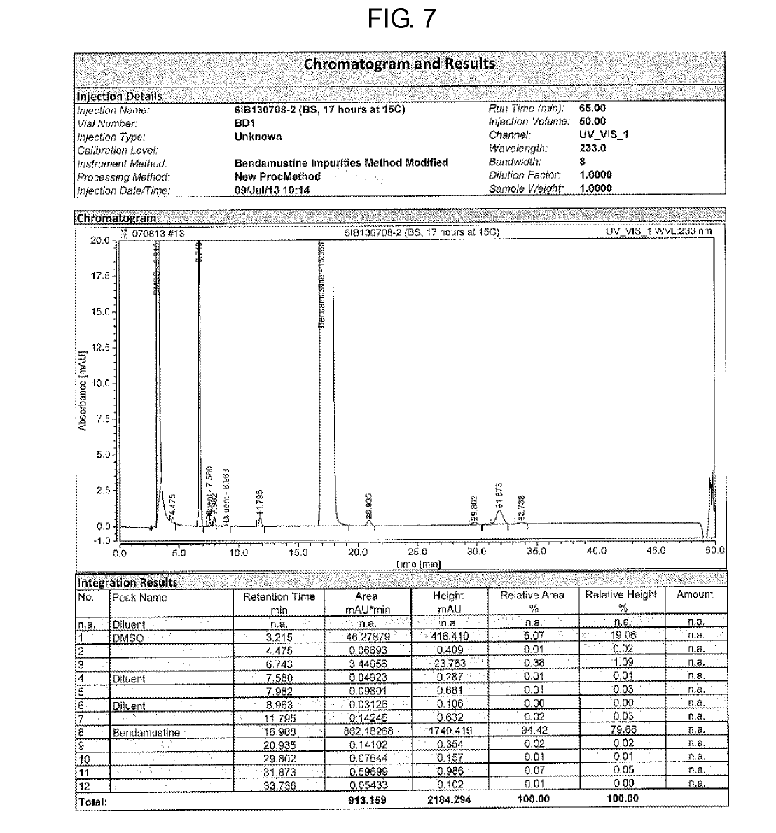

6IB130708-2/hold at 15° C. for 17 hrs (see e.g., Example 5, TABLE 4, FIG. 7). A 200 μg/ml solution of Bendamustine HCl in Diluent was prepared.

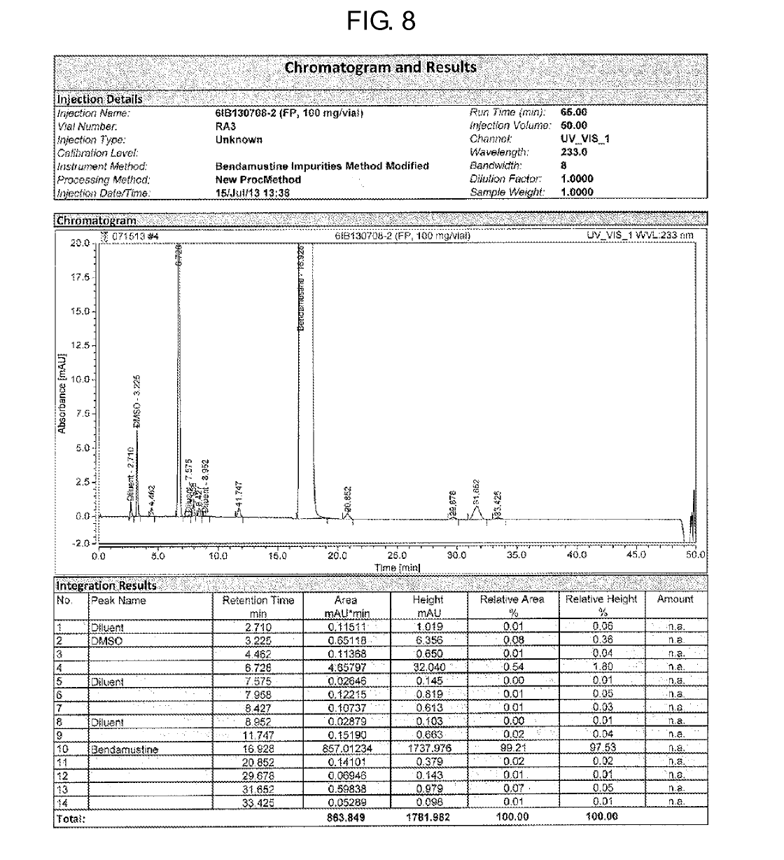

6IB130708-2 (lyophilized) 100 mg/vial (see e.g., Example 5, TABLE 4, FIG. 8). The final product was reconstituted with Diluent, and then a 200 μg/ml solution of Bendamustine HCl in Diluent was prepared.

6IB130709-2.

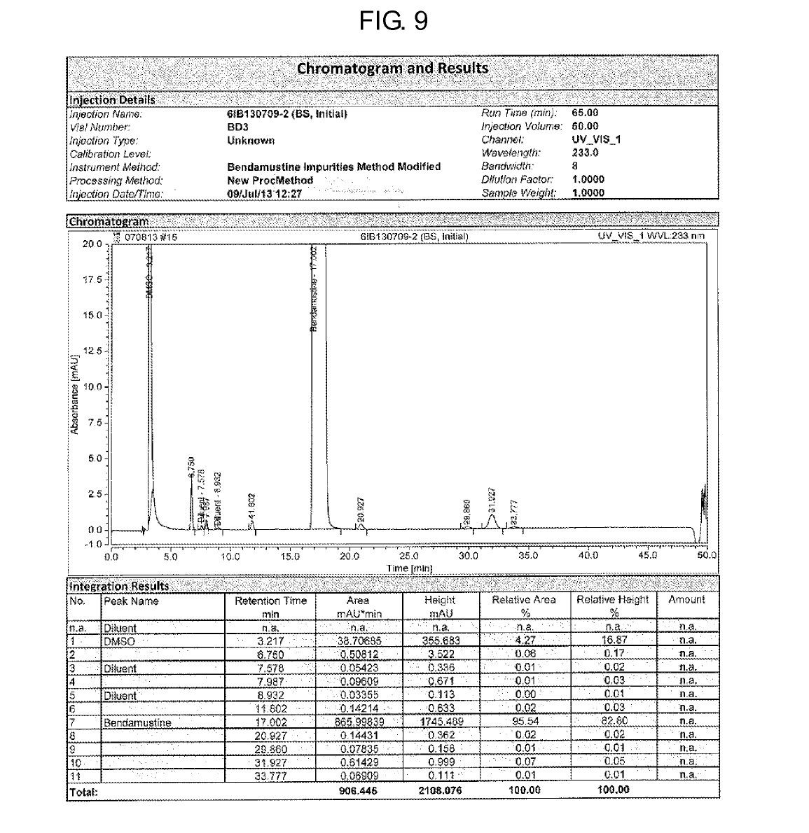

6IB130709-2 (40% DMSO/60% H₂O) compounded at 15° C. (see e.g., Example 7, TABLE 5, FIG. 9). A 200 μg/ml solution of Bendamustine HCl in Diluent was prepared.

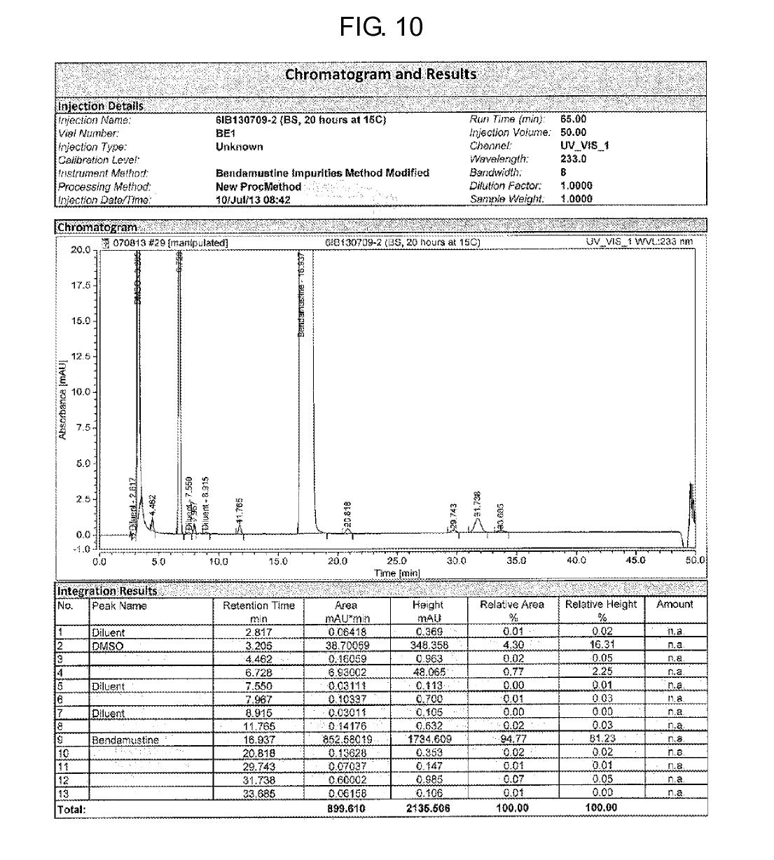

6IB130709-2 (40% DMSO/60% H₂O, 2 mg/ml) 20 hrs at 15° C., (see e.g., Example 7, TABLE 5, FIG. 10). A 200 μg/ml solution of Bendamustine HCl in Diluent was prepared.

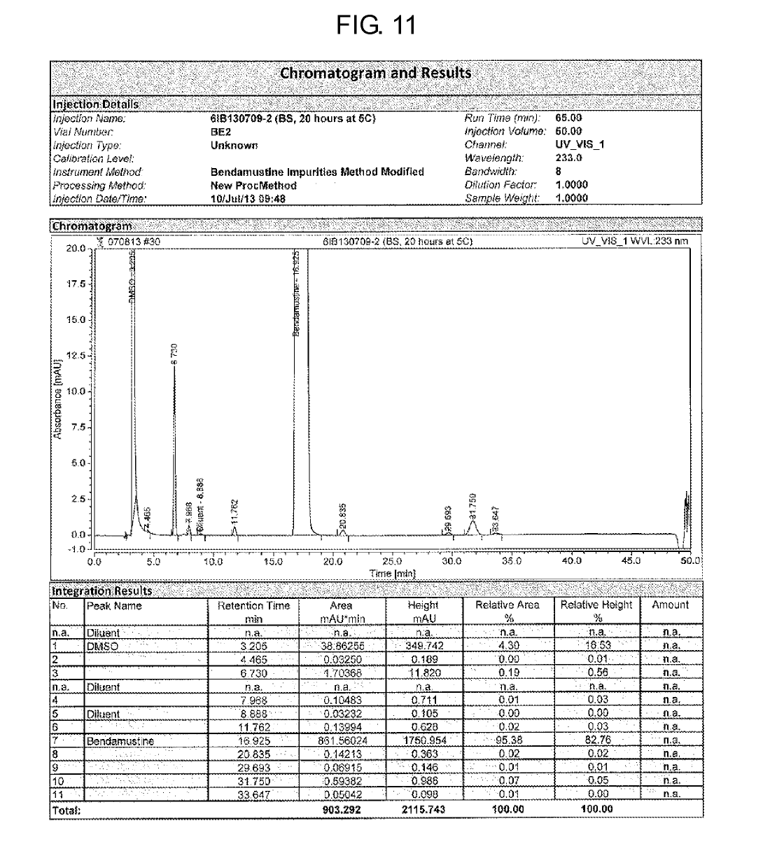

6IB130709-2 (40% DMSO/60% H₂O, 2 mg/ml), 20 hrs at 5° C. (see e.g., Example 7, TABLE 5, FIG. 11). A 200 μg/ml solution of Bendamustine HCl in Diluent was prepared.

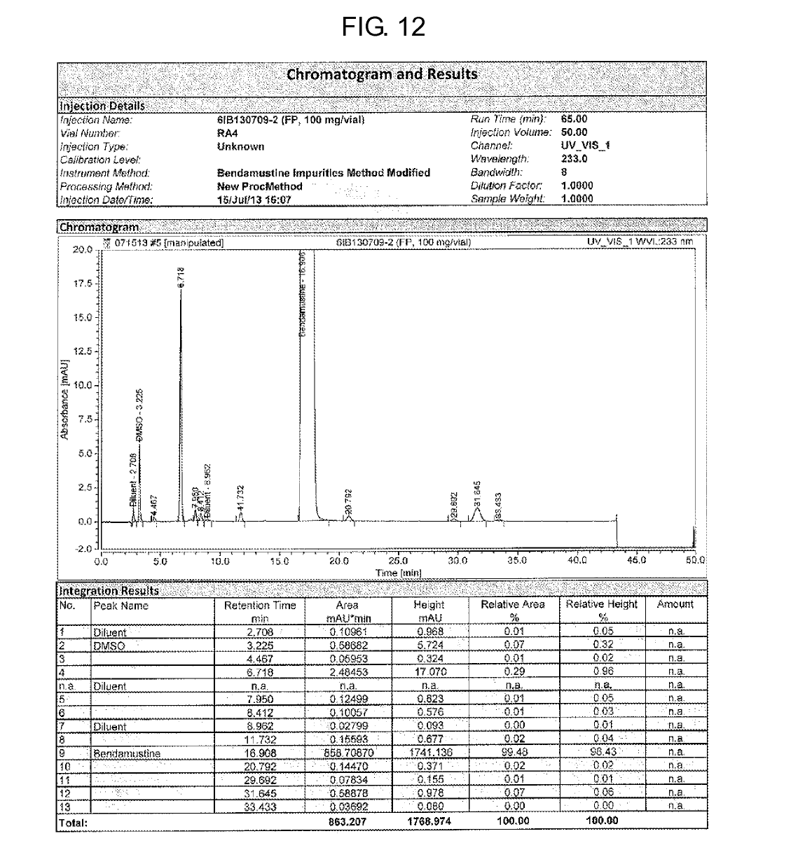

6IB130709-2 (lyophilized) 100 mg/vial (see e.g., Example 7, TABLE 5, FIG. 12). The final product was reconstituted with Diluent, and then a 200 μg/ml solution of Bendamustine HCl in Diluent was prepared.

### Example 4

### Formulation of Bendamustine (6IB130708-1)

The following Example describes a bendamustine formulation analyzed from the bulk solution, following storage, and the finished product. Methods are according to Examples 1-3 unless otherwise specified.

Each ml of the bulk solution contained: bendamustine hydrochloride (25 mg); mannitol (42.5 mg); and DMSO (q.s. to 1 ml). “q.s.”, is a Latin term for quantum sufficit, when used in chemistry means as much as suffices.

About 45 ml of DMSO was placed in a glass beaker. 2.1836 g of Mannitol was added and mixed at −350 rpm for 10 minutes at room temperature. 1.2582 g bendamustine was added to the beaker and mixed for 10 minutes. Bendamustine was completely dissolved after 5 minutes. The contents of the beaker was transferred to a volumetric flask and diluted to 50 ml with DMSO.

About 25 ml of the sample was placed at 5° C. and the rest at room temperature. After 10 minutes, the sample was frozen due to 100% v/v DMSO. The sample was removed from 5° C. and stored at 15° C., though further analysis was not performed.

The bulk solution that was stored at room temperature was analyzed further. The bulk solution was compounded and stored at room temperature (for 17 hours) prior to lyophilization. 4 ml of the batch solution was filled and lyophilized. Neither the compounding process nor the hold conditions significantly impacted impurity totals. Additional degradation following lyophilization was minor (see TABLE 3).

TABLE 3
Sample 61B130708-1 data of the initial Bulk Solution (see e.g.,
FIG. 3), Bulk Solution after a 17 hour hold time (see e.g.,
FIG. 4), and the Finished Product (see e.g., FIG. 5).

| | Sample | | |
|---|---|---|---|
| | | 6IB130708-1 | |
| | | Bulk Solution | 6IB130708-1 |
| | 6IB130708-1 | (Following | Finished |
| | Bulk Solution | hold at RT | Product |
| | (Initial) | for 17 hours) | (Initial) |
| | % | % | % |
| Impurity | Chromatographic | Chromatographic | Chromatographic |
| RRT | Area | Area | Area |
| 0.26 | N/A-DMSO* | N/A-DMSO* | |
| 0.40 | 0.04 | 0.03 | 0.04 |
| 0.47 | 0.01 | 0.01 | 0.07 |
| 0.50 | | | 0.01 |
| 0.69 | 0.01 | 0.02 | 0.02 |
| 1.23 | 0.02 | 0.02 | 0.02 |
| 1.76 | 0.01 | 0.01 | 0.01 |
| 1.88 | 0.06 | 0.06 | 0.06 |
| 1.99 | 0.01 | 0.01 | |
| Total % | 0.16 | 0.16 | 0.23 |
| Imp | | | |
| | | Reconstitution | 38 |
| | | Time (s) | |
| | | Residual Water | 0.77 |
| | | Content (%) | |

*The DMSO peak is disregarded from impurity totals.

### Example 5

### Formulation of Bendamustine (6IB130708-2)

The following Example describes a bendamustine formulation analyzed from the bulk solution, following storage, and the finished product. Methods are according to Examples 1-3 unless otherwise specified.

Each ml of the bulk solution contained: bendamustine hydrochloride (25 mg); mannitol (42.5 mg); DMSO (50% v/v in water) (q.s. to 1 ml).

25 ml of water was placed in a glass beaker and mix at about 350 rpm. 2.1624 g mannitol was added and mixed about 5 minutes. About 20 ml DMSO was added to the mannitol solution and temperature was brought to about 15° C. 1.2538 g bendamustine HCl was added and mixed. The above solution was transferred into a 50 ml volumetric flask and diluted to volume (50 ml) with DMSO.

About 25 ml of the bulk solution was placed in a 50 ml volumetric flask and stored at 15° C. The bulk solution was compounded and stored at 15° C. (for 17 hours) prior to lyophilization. Hydrolysis of bendamustine was observed to occur during the hold time. As such, for aqueous mixtures containing DMSO, compounding or holding at temperatures lower than 15° C. show favorable results. 4 ml of the batch solution was filled and lyophilized. Additional degradation following lyophilization was minor (see TABLE 4).

TABLE 4
Sample 61B130708-2 data of the initial Bulk Solution (see e.g.,
FIG. 6), Bulk Solution after a 17 hour hold time (see e.g.,
FIG. 7), and the Finished Product (see e.g., FIG. 8).

| | Sample | | |
|---|---|---|---|
| | | 6IB130708-2 | |
| | | Bulk Solution | 6IB130708-2 |
| | 6IB130708-2 | (Following | Finished |
| | Bulk Solution | hold at 15 C. | Product |
| | (Initial) | for 17 hours) | (Initial) |
| | % | % | % |
| Impurity | Chromatographic | Chromatographic | Chromatographic |
| RRT | Area | Area | Area |
| 0.26 | N/A-DMSO* | 0.01 | 0.01 |
| 0.40 | 0.09 | 0.38 | 0.54 |
| 0.47 | 0.01 | 0.01 | 0.01 |
| 0.50 | | | 0.01 |
| 0.69 | 0.02 | 0.02 | 0.02 |
| 1.23 | 0.02 | 0.02 | 0.02 |
| 1.76 | 0.01 | 0.01 | 0.01 |
| 1.88 | 0.07 | 0.07 | 0.07 |
| 1.99 | 0.01 | 0.01 | 0.01 |
| Total % | 0.23 | 0.53 | 0.70 |
| Imp | | | |
| | | Reconstitution | 37 |
| | | Time (s) | |
| | | Residual Water | 0.83 |
| | | Content (%) | |

*The DMSO peak is disregarded from impurity totals.

### Example 6

### Formulation of Bendamustine (6IB130709-1)

The following Example describes a bendamustine composition of sample 6IB130709-1. Methods are according to Examples 1-3 unless otherwise specified.

Each ml of the bulk solution contained: bendamustine hydrochloride (25 mg); mannitol (42.5 mg); DMSO (25% v/v in water) (q.s. to 1 ml).

37.5 ml of water was placed in a glass beaker and mixing was initiated at about 350 rpm. 2.1266 g mannitol added and mixed for about 5 minutes. About 12.5 ml of DMSO was added and solution brought to about 15° C. 1.2672 g of bendamustine HCl added and mixed. A milky solution was the result. Bendamustine HCl was not found to be soluble in 75% water.

The sample was not used for analysis purposes, however, 3 ml of DMSO was added and the sample was cloudy. Another 3 ml of DMSO was added and the solution started to clear up. The addition of DMSO corresponds to about 35% DMSO. This suggests that more than about 35% DMSO solution is optimal for bendamustine dissolution at 15° C.

### Example 7

### Formulation of Bendamustine (6IB130709-2)

The following Example describes a bendamustine composition of sample 6IB130709-2, analyzed from the bulk solution, following storage at two different temperatures, and the finished product. Methods are according to Examples 1-3 unless otherwise specified.

Each ml of the bulk solution contained: bendamustine hydrochloride (25 mg); mannitol (42.5 mg); DMSO (40% v/v in water) (q.s. to 1 ml).

30 ml of water was placed in a glass beaker and mixing was initiated at about 350 rpm. 2.1268 g mannitol was added and mixed for about 5 min. About 17 ml of DMSO was added and the temperature was brought to about 15° C. 1.2535 g bendamustine HCl was added and mixed. The solution was transferred into a 50 ml volumetric flask and diluted to volume with DMSO.

About 5 ml was placed at 5° C. and another 5 ml at 15° C. The bulk solution was compounded at 15° C. Following compounding, the bulk solution was split and held at two different temperatures (15° C. and 5° C.) for 20 hours prior to lyophilization. For the portion held at 15° C., the level of bendamustine hydrolysis was high. However, the portion held at 5° C. showed much less hydrolytic degradation. As such, for aqueous mixtures containing DMSO, compounding or holding at temperatures lower than 15° C. show favorable results. 4 ml of the batch solution was filled and lyophilized. Only the portion held at 5° C. was filled and lyophilized. Additional degradation following lyophilization was minor (see TABLE 5).

TABLE 5
Sample 61B130709-2 data of the initial Bulk Solution (see e.g., FIG. 9),
Bulk Solution after a 20 hour hold time at 15° C. (see e.g., FIG. 10)
and 5° C. (see e.g., FIG. 11), and the Finished Product (see e.g., FIG. 12).

| | Sample | | | |
|---|---|---|---|---|
| | | 6IB130709-2 | 6IB130709-2 | |
| | | Bulk Solution | Bulk Solution | 6IB130709-2 |
| | 6IB130709-2 | (Following | (Following | Finished |
| | Bulk Solution | hold at 15 C. | hold at 5 C. | Pr [symbol] |
| | (Initial) | for 20 hours) | for 20 hours) | (Initial) |
| | % | % | % | % |
| Impurity | Chromatographic | Chromatographic | Chromatographic | Chromatographic |
| RRT | Area | Area | Area | Area |
| 0.26 | N/A-DMSO* | 0.02 | | 0.0 [symbol] |
| 0.40 | 0.05 | 0.77 | 0.19 | 0.29 |
| 0.47 | 0.02 | 0.01 | 0.01 | 0.0 [symbol] |
| 0.50 | | | | 0.0 [symbol] |
| 0.69 | 0.02 | 0.02 | 0.02 | 0.0 [symbol] |
| 1.23 | 0.01 | 0.02 | 0.02 | 0.0 [symbol] |
| 1.75 | 0.02 | 0.01 | 0.01 | 0.0 [symbol] |
| 1.88 | 0.07 | 0.07 | 0.07 | 0.07 |
| 1.99 | 0.01 | 0.01 | 0.01 | |
| Total % | 0.20 | 0.93 | 0.33 | 0.4 [symbol] |
| Imp | | | | |
| | | | Reconstitution | [symbol] |
| | | | Time (s) | |
| | | | Residual Water | [symbol] |
| | | | Content (%) | |

*The DMSO peak is disregarded from impurity totals. [symbol]
indicates data missing or illegible when filed

### Example 8

### Percent Water Analysis and Reconstitution of Lyophilized Bendamustine

The following Example describes the percent water content and reconstitution time of lyophilized bendamustine samples: 6IB130708-1 (see Example 4); 6IB130708-2 (see Example 5); 6IB130709-2 (see Example 7).

Methods are according to Examples 1-3 unless otherwise specified. Results are reported in TABLE 6.

TABLE 6
Data of reconstitution time (after 20 ml water for injection USP
was added to each vial and shaken), description of lyophilized sample,
measured weight of the sample, and % H₂O of the sample.

| | Recon- | | After | | |
|---|---|---|---|---|---|
| | stitution | Gross | dis- | Actual | |
| Sample ID | Time | wt | pensing | wt | H₂O |
| 6IB130708-1 | 38 sec | 3.9612 g | 3.8646 g | 0.0966 g | 0.7696% |
| (100% DMSO) | | | | | |
| 6IB130708-2 | 37 sec | 4.0371 g | 3.9568 g | 0.0803 g | 0.8284% |
| (50:50 | | | | | |
| DMSO H₂O) | | | | | |
| 6IB130709-2 | 43 sec | 4.1439 g | 4.0850 g | 0.0589 g | 0.8514% |
| (40:60 | | | | | |
| DMSO:H₂O) | | | | | |

### Example 9

### Lyophilization

The following Example describes a lyophilization protocol used in Examples 10-16.

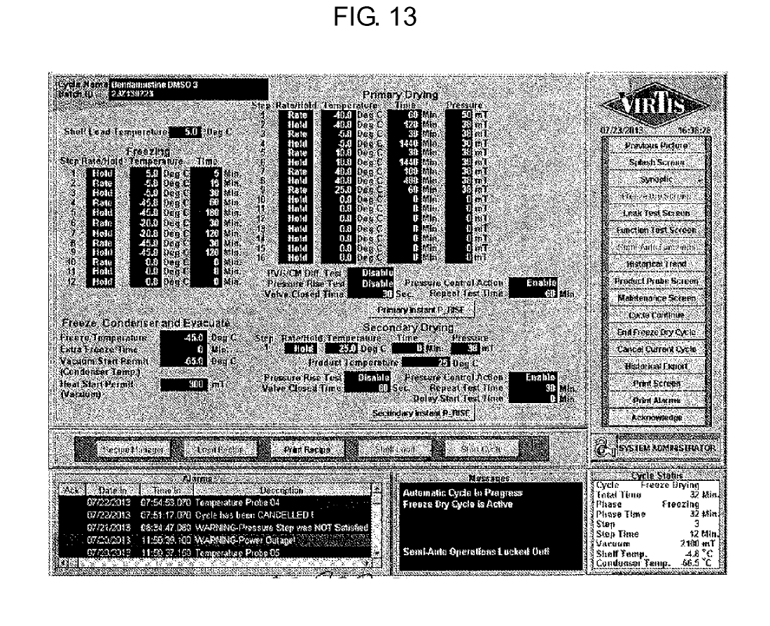

The freeze dryer used in Examples 10-16 was a Virtis Model (Genesis 25 xL). Parameters used for freeze drying can be found in FIG. 13.

A 25 mg/ml bulk solution of lab batch 2JZ130723P16 was prepared according to the following: 2.1327 g mannitol was added to a 100 ml glass beaker. 40 ml of 100% DMSO was added to the beaker and stirred with a stir bar at room temperature and 300 rpm for 10 min resulting in a clear solution. 1.2510 g bendamustine was added to the beaker and the weighing boat was rinsed into the beaker with 5 ml DMSO and stirred with a stir bar at room temperature and 300 rpm for 5 minutes resulting in a clear solution. The solution was diluted to 50 ml in a volumetric flask resulting in a bulk solution of 25 mg/ml bendamustine and 42.5 mg/ml of mannitol.

The 25 mg/ml bulk solution of lab batch 2JZ130723P16 was lyophilized according to the lyophilization cycle presented below:

TABLE 7
A preferred lyophilization cycle recipe for
bendamustine HCl for injection (100 mg/vial)

| | | Rate or | Temp | Time | Vacuum |
|---|---|---|---|---|---|
| Step | | Hold Step | (° C.) | (hr) | (mT) |
| 1. | Loading | Hold | 5 | 0.083 | — |
| 2. | Freezing | Rate | −5 | 0.250 | — |
| 3. | Freezing | Hold | −5 | 0.500 | — |
| 4. | Freezing | Rate | −45 | 1.000 | — |
| 5. | Freezing | Hold | −45 | 3.000 | — |
| 6. | Annealing | Rate | −20 | 0.500 | — |
| 7. | Annealing | Hold | −20 | 2.000 | — |
| 8. | Freezing | Rate | −45 | 0.500 | — |
| 9. | Freezing | Hold | −45 | 2.000 | — |
| 10. | Primary drying | Rate | −40 | 1.000 | 50 |
| 11. | Primary drying | Hold | −40 | 2.000 | 38 |
| 12. | Primary drying | Rate | −5 | 0.500 | 38 |
| 13. | Primary drying | Hold | −5 | 24.000 | 38 |
| 14. | Primary drying | Rate | 10 | 0.500 | 38 |
| 15. | Primary drying | Hold | 10 | 24.000 | 38 |
| 16. | Secondary drying | Rate | 40 | 3.000 | 38 |
| 17. | Secondary drying | Hold | 40 | 8.000 | 38 |
| 18. | Secondary drying | Rate | 25 | 1.000 | 38 |

| | | | | |
|---|---|---|---|---|
| Total Cycle Time (hr) | — | — | 73.833 | — |

Following lyophilization, the sample contained about 100 mg/vial Bendamustine HCl, about 170 mg/vial Mannitol, and trace amounts of DMSO and water.

### Example 10

### Impurity Analysis

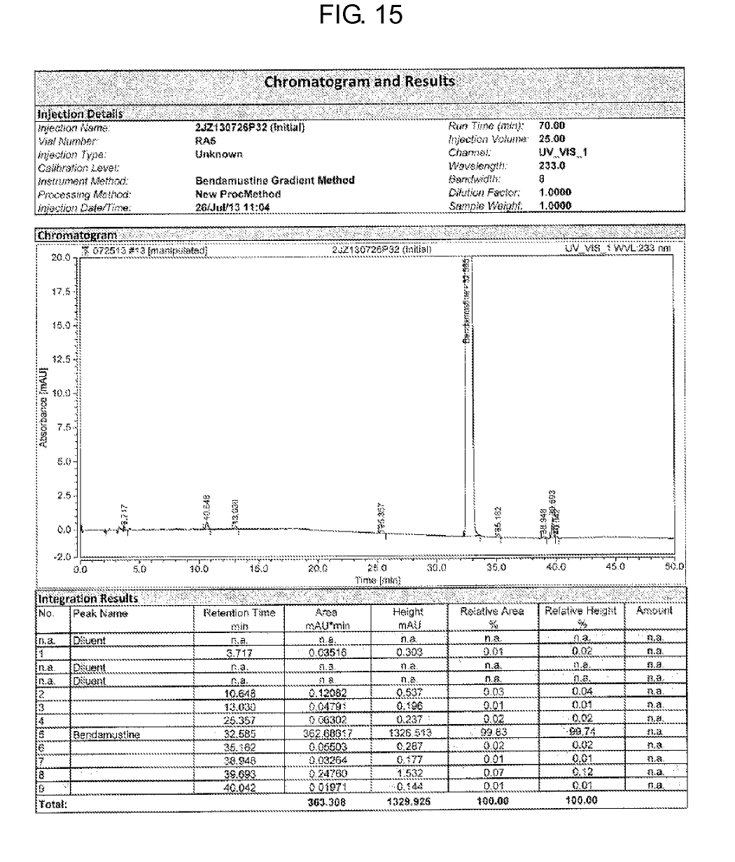

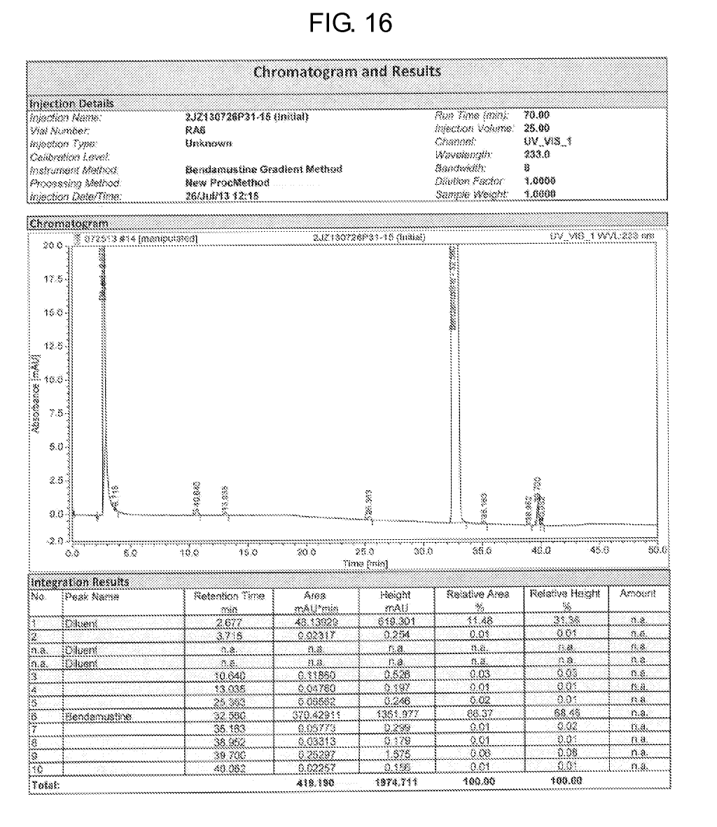

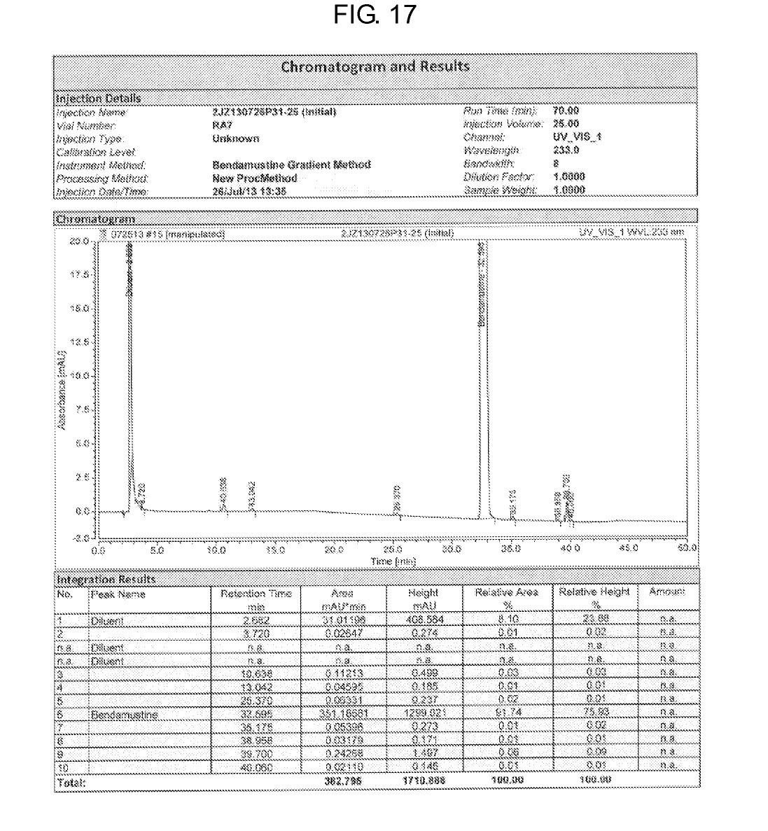

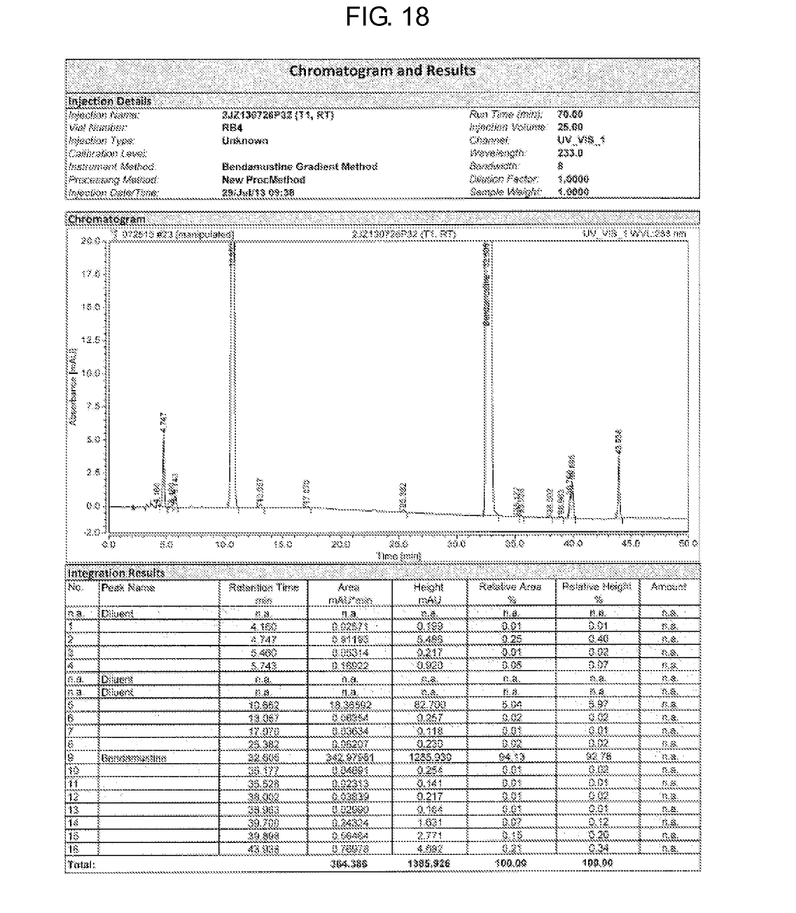

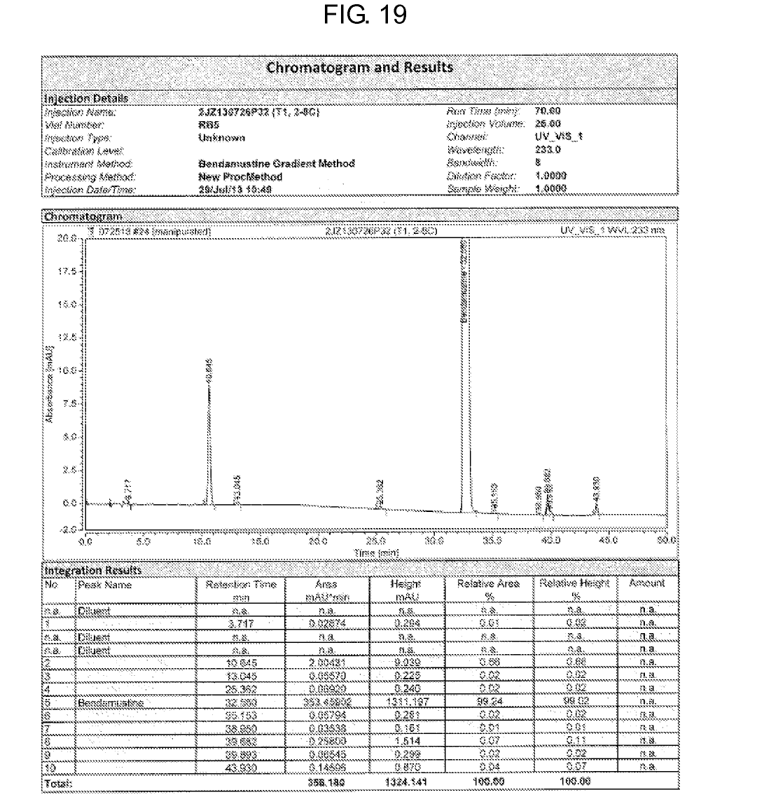

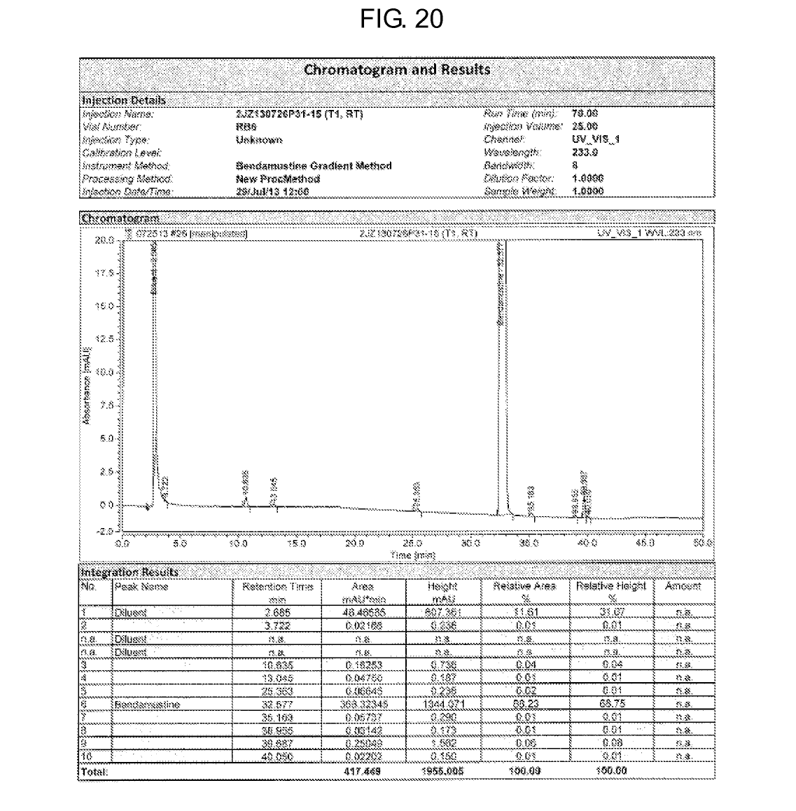

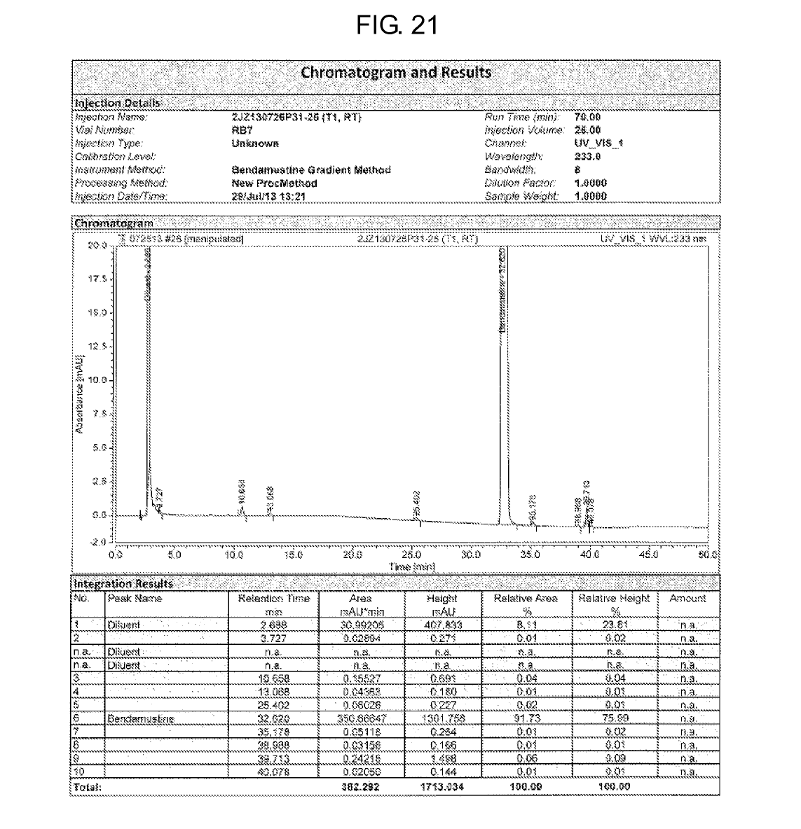

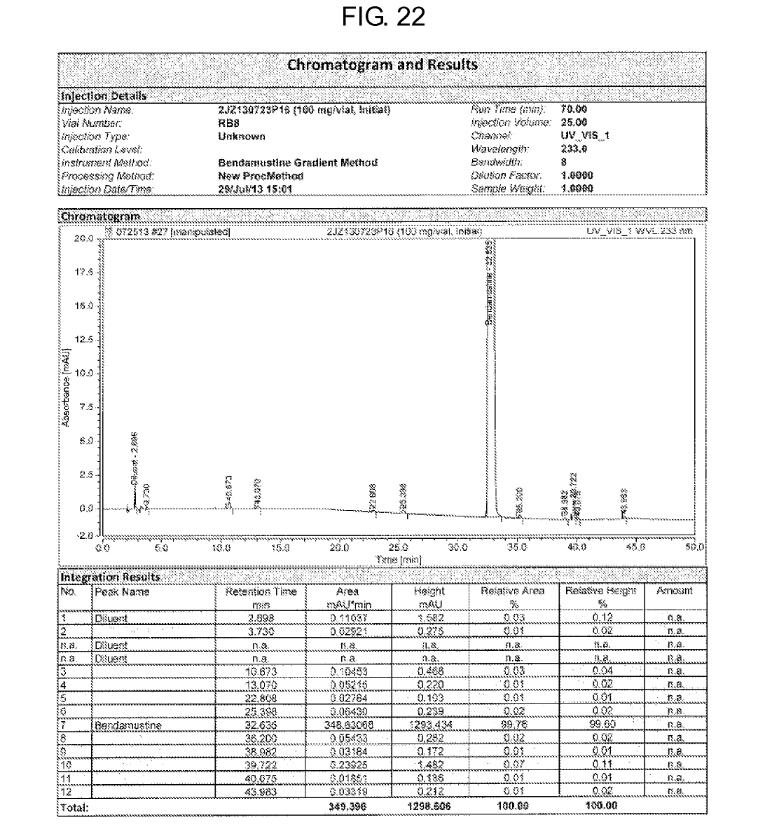

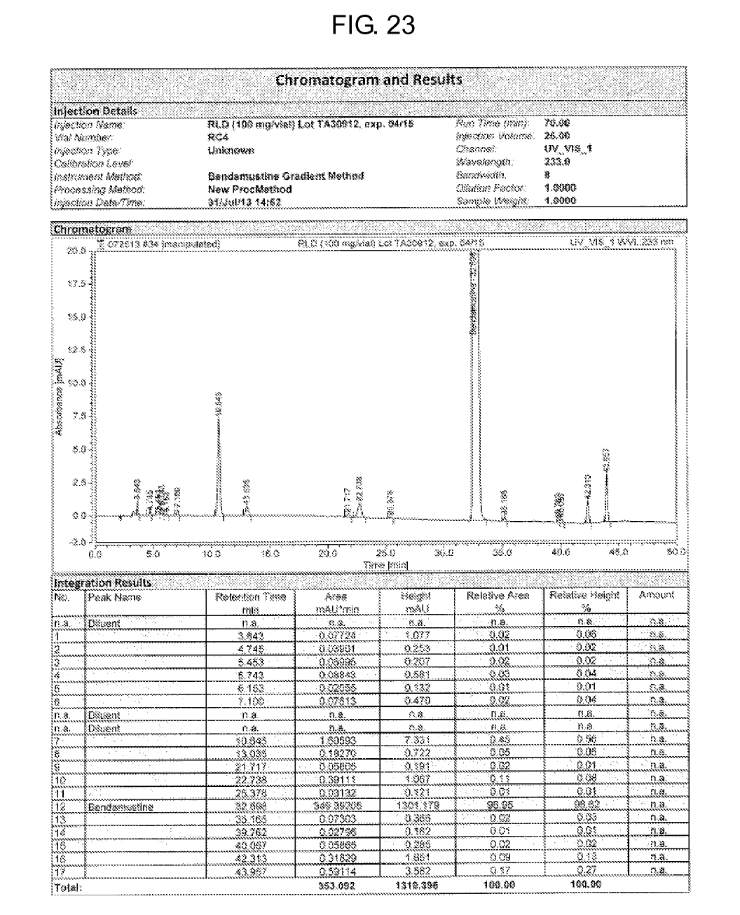

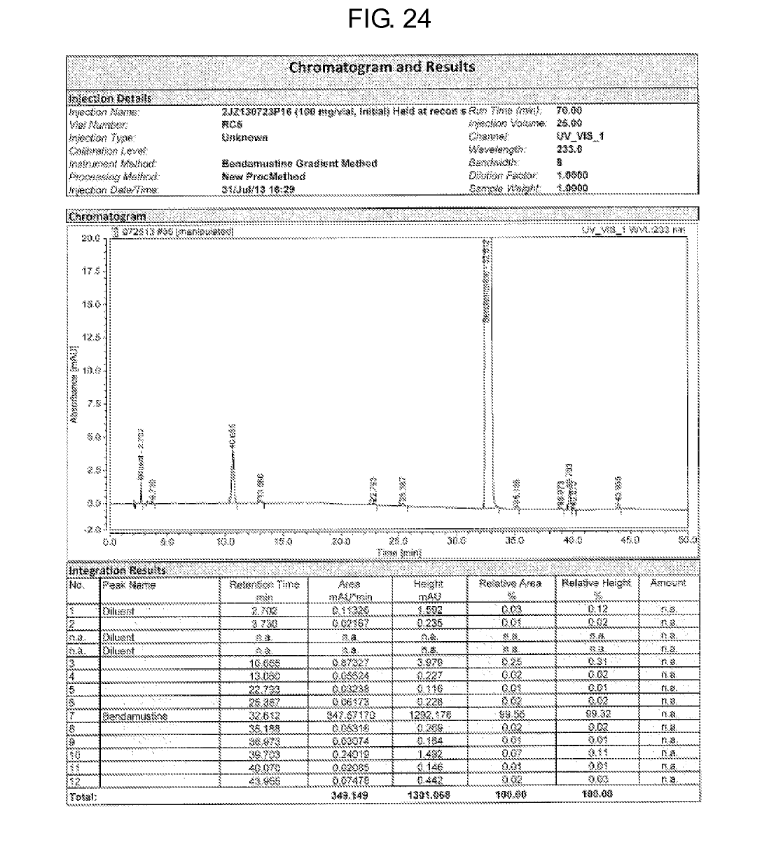

The following Example describes procedure and chromatogram results for 072513 (see e.g., FIG. 15, FIG. 16, FIG. 17, FIG. 18, FIG. 19, FIG. 20, FIG. 21, FIG. 22, FIG. 23, and FIG. 24) impurity analysis sequence.

Mobile Phase A: 10 mM Potassium Hexafluorophosphate buffer, pH 3.0. Mobile Phase B: HPLC-grade Acetonitrile. Diluent: a 60:40 mixture of Mobile Phase A and Mobile Phase B. Impurity standard: Bendamustine HCl was exposed for 1 hour at room temperature under bench to achieve H₂O saturation. A solution of 2 μg/ml Bendamustine HCl in Diluent was prepared.

The LC protocol is as follows:

Instrument: Dionex UltiMate 3000 equipped with a UV Detector and cooled Autosampler. Column: Shim-pack VP-ODS 250 L×4.6 mm, 5 μm. Flow Rate: about 1.0 ml/minute. Detector Wavelength: 233 nm, 8 nm bandwidth. Injection volume: 25 μl. Column Temperature: 25° C. Autosampler Tray Temperature: 5° C.

TABLE 8
Gradient

| Time [min] | Mobile Phase B (%) |
|---|---|
| 0.0 | 30.0 |
| 15.0 | 30.0 |
| 40.0 | 50.0 |
| 50.0 | 50.0 |
| 50.1 | 90.0 |
| 60.0 | 90.0 |
| 60.1 | 30.0 |
| 70.0 | 30.0 |

Samples for impurity analysis were prepared as follows.

2JZ130726P31-25 (bulk solution (BS)=25 mg/ml; 100% DMSO) compounded at 25° C. (see e.g., FIG. 17, Example 12). A solution of 200 μg/ml Bendamustine HCl in Diluent was prepared.

2JZ130726P31-25 (bulk solution (BS)=25 mg/ml; 100% DMSO) held at 25° C. for 72 hours (see e.g., FIG. 21, Example 12). A solution of 200 μg/ml Bendamustine HCl in Diluent was prepared.

2JZ130726P31-15 (bulk solution (BS)=15 mg/ml, 100% DMSO) compounded at 25° C. (see e.g., FIG. 16, Example 11). A solution of 210 μg/ml Bendamustine HCl in Diluent was prepared.

2JZ130726P31-15 (bulk solution (BS)=15 mg/ml, 100% DMSO) held at 25° C. for 72 hours (see e.g., FIG. 20, Example 11). A solution of 210 μg/ml Bendamustine HCl in Diluent was prepared.

2JZ130726P32 (bulk solution (BS)=15 mg/ml, 30% TBA) compounded at 5° C. (see e.g., FIG. 15, Example 11). A solution of 210 μg/ml Bendamustine HCl in Diluent was prepared.

2JZ130726P32 (bulk solution (BS)=15 mg/ml, 30% TBA) held at 25° C. for 72 hours (see e.g., FIG. 18, Example 11). A solution of 210 μg/ml Bendamustine HCl in Diluent was prepared.

2JZ130726P32 (bulk solution (BS)=15 mg/ml, 30% TBA) held at 5° C. for 72 hours (see e.g., FIG. 19, Example 11). A solution of 210 μg/ml Bendamustine HCl in Diluent was prepared.

Reference Listed Drug (RLD) (Treanda®) Sample Preparation (100 mg/vial, see e.g., FIG. 23). The RLD was reconstituted with Water for Injection, and then a solution of 200 μg/ml Bendamustine HCl in Diluent was prepared.

2JZ130723P16 (100 mg/vial, Initial) (see e.g., FIG. 22, TABLE 13, Example 13, Example 16). The final product with Diluent was reconstituted, and then a solution of 200 μg/ml Bendamustine HCl in Diluent was prepared.

2JZ130723P16 (100 mg/vial, Initial, Held at Reconstitution Step) (see e.g., FIG. 24, TABLE 13, Example 13, Example 16). The final product was reconstituted with Water for Injection. The mixture was left for 2.5 min to mimic reference listed drug (RLD) reconstitution time. The actual reconstitution time of 2JZ130723P16 was observed to be 30 s. A solution of 200 μg/ml Bendamustine HCl in Diluent was prepared.

### Example 11

### Bulk Solution Hold Time Study A

The following Example describes the study of the bulk solution (pre-lyophilization composition) hold time for Treanda® (batch 2JZ130726P32) and bendamustine test formulation (batch 2JZ130726P31-15). Methods are according to Examples 1, 9, and 10 unless otherwise specified.

A bulk solution stability comparison study was carried out by preparing both the Treanda®, the reference listed drug (RLD), formulation as described in U.S. Pat. No. 8,436,190 and the test formulation at identical active pharmaceutical ingredient (API)/mannitol concentrations. A summary of the two lab formulations is provided below in TABLE 9.

The preparation of batch 2JZ130726P31-15 was prepared as follows: 2.1370 g mannitol was added to a 100 ml glass beaker. 40 ml of DMSO was added to the beaker and stirred with a stir bar at room temperature and 300 rpm for 10 min. 1.2583 g bendamustine was added to the beaker and the weighing boat was rinsed into the beaker with 5 ml DMSO and stirred with a stir bar at room temperature and 300 rpm for 10 minutes. The solution was diluted to 50 ml in a volumetric flask resulting in a composition described in TABLE 11. This bulk solution was then diluted to by removing 15 ml of the above bulk solution and diluting to 25 ml with DMSO in a volumetric flask, resulting in a concentration of 15 mg/ml.

The preparation of Treanda® batch 2JZ130726P32 formulation with 30% TBA was prepared as follows: 1.2775 g mannitol was added to a 100 ml jacketed beaker with 30 ml MilliQ water and stirred at room temperature and 300 rpm with a stir bar for 10 minutes. 15 ml TBA was added to the above solution and stirred at room temperature and 300 rpm. The jacket was connected to a circulator cooled to 5° C. and stirred at 150 rpm with a stir bar and the circulator was adjusted to 4° C. 0.75 g Bendamustine HCl was added to the beaker. The solution was stirred at 300 rpm with circulator to maintain at 5° C. for 10 min. The measured temperature was 5.1° C. MilliQ water was added to reach a final volume of 50 ml in a 50 ml volumetric flask.

TABLE 9
Summary of lab formulations in different conditions.

| | | | Test |
|---|---|---|---|
| | | RLD | Formulation |
| | | Treanda ® | (Lot No. |
| | | Formulation (Lot | 2JZ130726P31- |
| | | No. 2JZ130726P32) | 15) |
| Bulk Solution | Bendamustine | 15 mg/ml | 15 mg/ml |
| Composition | Hydrochloride | | |
| | Mannitol, USP | 25.5 mg/ml | 25.5 mg/ml |
| | q.s. to 1 ml | 30% TBA¹ | 100% DMSO² |
| | | v/v in Water | |
| Processing | Compounding | 5° C. | 25° C. |
| Conditions | Temperature | | |
| | Hold at 5° C. | Yes | N/A |
| | for 72 hrs | | |
| | Hold at 25° C. | Yes | Yes |
| | for 72 hrs | | |

¹TBA = tert-Butyl Alcohol
²DMSO = Dimethyl Sulfoxide

For both of the lab batches, impurity content was determined before and after the respective hold periods. T=72 data was not generated for the test formulation at 5° C. as the bulk solution would freeze at 5° C. in the presence of 100% DMSO (see N/A in TABLE 9). The total impurities data is provided in TABLE 10.

TABLE 10
Summary of Impurities.

| | Treanda ® | | | |
|---|---|---|---|---|
| | Formulation | | Test Formulation | |
| Hold | Total Imp. % | Total Imp. % | Total Imp. % | Total Imp. % |
| Temperature | T = 0 hr | T = 72 hr | T = 0 hr | T = 72 hr |
| 5° C. | 0.18 | 0.77 | 0.16 | N/A |
| 25° C. | 0.18 | 5.88 | 0.16 | 0.17 |

While both formulations demonstrated comparable total impurity levels immediately after compounding (T=0), only the test formulation was found to be stable with no increase in total degradation products over the 72-hour hold period. On the other hand, the reference listed drug (RLD) formulation bulk showed a significant increase in total degradation products at both 5° C. and 25° C. after 72 hrs (0.77% and 5.88%, respectively).

Compared to the Treanda® formulation, the enhanced stability of the test formulation was attributed to the elimination of water as a formula ingredient in the bulk solution. Consequently, the 72-hour stability at room temperature provides greater manufacturing flexibility than the Treanda® formulation, which must be maintained at low temperatures and has a limited hold time.

### Example 12

### Optimization Study A

The following Example describes the optimization of a bendamustine composition to increase hold time. Methods are according to Examples 1, 9, and 10 unless otherwise specified.

While the bulk hold time study was carried out at a concentration similar to the reference listed drug (RLD) product (see Example 11), test formulations were further optimized to reduce the DMSO content in the bulk by increasing the concentrations of bendamustine and mannitol. Thus a lab batch, 2JZ130726P31-25, was formulated (see TABLE 11).

Lab batch, 2JZ130726P31-25 was prepared according to the following: 2.1370 g mannitol was added to a 100 ml glass beaker. 40 ml of DMSO was added to the beaker and stirred with a stir bar at room temperature and 300 rpm for 10 min. 1.2583 g bendamustine was added to the beaker and the weighing boat was rinsed into the beaker with 5 ml DMSO and stirred with a stir bar at room temperature and 300 rpm for 10 minutes. The solution was diluted to 50 ml in a volumetric flask resulting in a composition described in TABLE 11.

TABLE 11
Batch 2JZ130726P31-25 composition.

| | Test Formulation |
|---|---|
| | -per ml- |
| Bulk Solution Composition | (Lot No. 2JZ130726P31-25) |
| Bendamustine Hydrochloride | 25 mg |
| Mannitol, USP | 42.5 mg |
| q.s. to 1 ml | DMSO (100%) |

The modified lab batch was compounded and held at 25° C. for 72 hours, confirming the higher bulk solution concentrations did not impact degradation. Identical stability and degradation profile was observed when compared with the lower concentration bulk.

TABLE 12
Total Impurities at 0 hr and 72 hr at 25° C.

| | Test Formulation | Test Formulation |
|---|---|---|
| Hold | T = 0 hr | T = 72 hr |
| Temperature | (Total Imp. %) | (Total Imp. %) |
| 25° C. | 0.16 | 0.17 |

Impurity data confirmed the higher bulk solution concentrations did not cause degradation, even after the 72-hour hold period. High bulk solution concentrations reduce the overall liquid fill volume that can be removed via lyophilization. This can result in shorter cycle times and reduced operating costs, as well as added safety benefits due to potentially lower residual solvents present in the finished product.

### Example 13

### Percent Water Analysis and Reconstitution of Lyophilized Bendamustine

The following Example describes the percent water content and reconstitution of lyophilized bendamustine (see TABLE 13). Methods are according to Examples 1, 9, and 10 unless otherwise specified.

The reconstitution for RLD (Treanda®, bendamustine HCl for injection, Cephalon, Lot # TA 30912, 2JZ130726P32, 100 mg/vial,) was performed by injecting 5 ml sterile water for injection, usp, with a BD 5 ml syringe and a 18 g 1% needle and shaken by hand for 2 minutes 30 seconds for a result of a clear solution.

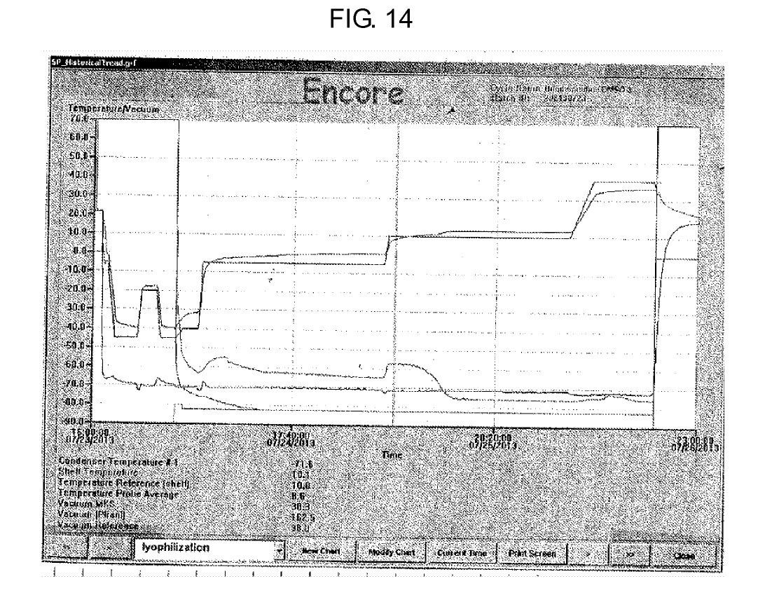

The reconstitution for lab batch 2JZ130723P16 was performed by injecting 20 ml of MilliQ water with a syringe/needle 18 g 1½ and shaken by hand for 20 seconds. The pH of the reconstituted lab batch was 3.11. The graph of the lyophilization time vs. temperature plot is shown in FIG. 14.

TABLE 13
The physical and chemical properties for the lyophilized
lab batch.

| Analytical Test | 2JZ130723P16 | | Treanda ® RLD |
|---|---|---|---|
| Appearance of Lyo | A white cake | | A white to |
| | | | off-white powder |
| % Total Impurities | 0.20¹ | 0.44² | 1.06² |

| | | |
|---|---|---|
| Reconstitution Time (s) | 20 | 150 |
| Residual Water Content (%) | 1.0 | 0.5³ |
| Residual DMSO Content (mg/vial) | 8.1 | N/A |

¹Test sample prepped for impurity analysis as per analytical test method.
²Test sample was reconstituted with Water for Injection (WFI), held for the same amount of time as the reference listed drug (RLD) reconstitution time, then prepped for impurity analysis. Reference listed drug (RLD) sample (100 mg/vial, Lot TA30912, exp. April 2015) first reconstituted with WFI and then prepped for impurity analysis.
³Reference listed drug (RLD) samples prepared from 25 mg/vial (Lot TB30612, exp. February 2015).

Compared to impurity levels of the analogous bulk solution (Lot No. 2JZ130726P31-25), the lyophilization cycle did not impact the purity of the resulting product. Furthermore, the test formulation contained lower total degradation products than the reference listed drug (RLD), Treanda® (0.44% and 1.06%, respectively). The reconstitution time (20 s) was significantly lower than the reconstitution time of the reference listed drug (RLD) (150 s). Additionally, the trace level of residual water was typical of other lyophilized products and consistent with data for the reference listed drug (RLD). The residual level of the class 3 Solvent DMSO measured in the finished product is less than the 50 mg per day limit established in ICH Q3C.

### Example 14

### Formulation of Bendamustine (2JZ130731P47)

The following Example describes a formulation of bendamustine with 100% DMSO at higher concentration. Methods are according to Examples 1, 9, and 10 unless otherwise specified.

A 50 mg/ml bulk solution of lab batch 2JZ130731P47 was prepared according to the following: 4.2544 g mannitol was added to a 150 ml glass beaker. 40 ml of 100% DMSO was added to the beaker and stirred with a stir bar at room temperature and 400 rpm for 10 min. 2.5108 g bendamustine HCl was added to the beaker and the weighing boat was rinsed into the beaker with 5 ml DMSO and stirred with a stir bar at room temperature and 400 rpm for 10 minutes. The beaker was covered with aluminum foil. The solution was diluted to 50 ml in a volumetric flask resulting in a bulk solution of 50 mg/ml bendamustine and 85 mg/ml of mannitol.

### Example 15

### Water Content Determination

The following Example describes the determination of water content in batch samples: 2JZ130723P16. Methods are according to Examples 1, 9, and 10 unless otherwise specified.

The water content was determined by Karl Fisher Coulometric Titration. Aquastar Combi Coulomet Fritless reagent was used. The parameters include an extraction time of 120 seconds and a 45% mixing speed.

The total weight dispensed for batch 2JZ130723P16 was 0.0863 g. The % water for batch 2JZ130723P16 was determined to be 0.9739%.

### Example 16

### Residual DMSO Content Analysis

The following Example describes the DMSO content analysis using liquid chromatography (LC). Methods are according to Examples 1, 9, and 10 unless otherwise specified.

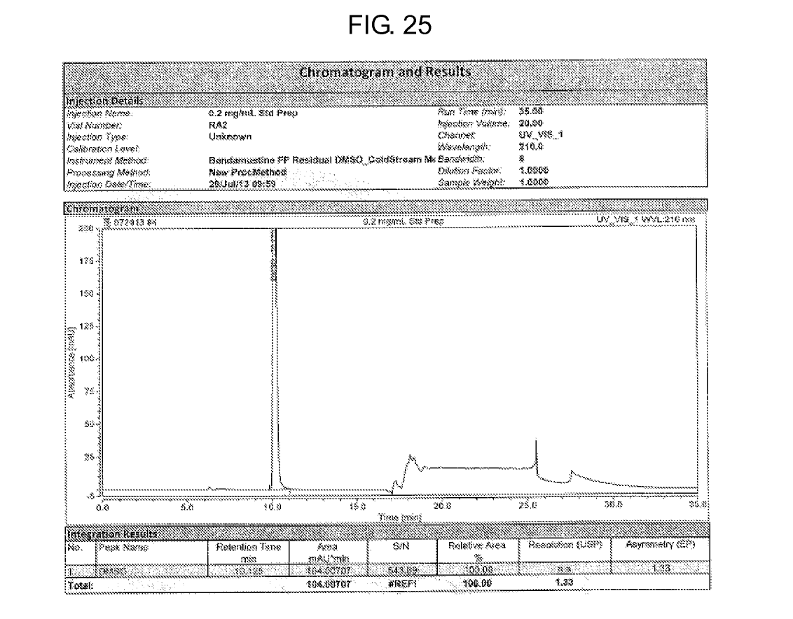

Mobile Phase A: 10 mM Potassium Dihydrogen Orthophosphate buffer. Mobile Phase B: HPLC-grade Acetonitrile. Diluent: 10 mM Disodium Hydrogen Orthophosphate buffer, pH 6.5. DMSO standard: a solution of 0.2 mg/ml DMSO in Diluent was prepared (see e.g., FIG. 25).

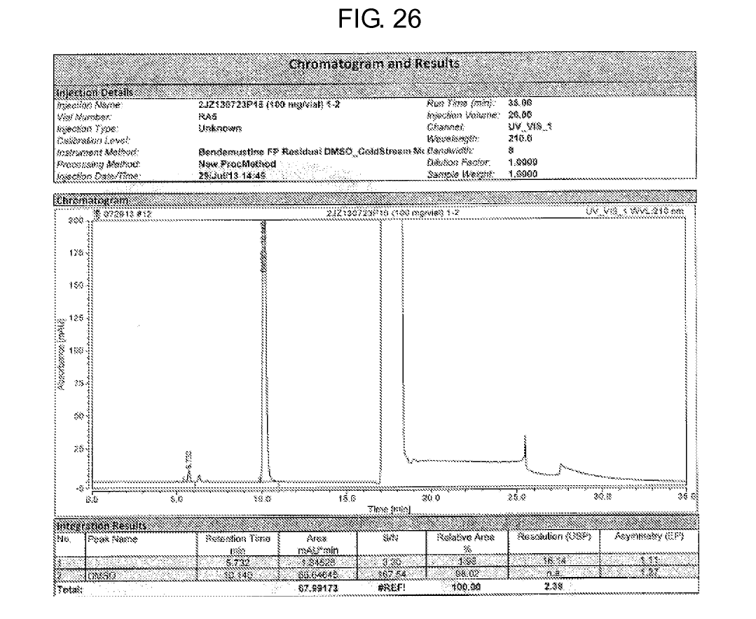

The liquid chromatography (LC) protocol for the 072913 sequence (see e.g., FIG. 25-26) is as follows.

Instrument: Dionex UltiMate 3000 equipped with a UV Detector and cooled Autosampler. Column: Intertsil ODS-3, 5 μm, 4.6×250 mm.

Flow Rate: about 0.5 ml/minute. Detector Wavelength: 210 nm, 8 nm bandwidth. Injection volume: 20 μl. Column Temperature: 30° C. Autosampler Tray Temperature: 5° C.

TABLE 14
Gradient

| Time [min] | Mobile Phase B (%) |
|---|---|
| 0.0 | 0.0 |
| 10.0 | 0.0 |
| 10.1 | 80.0 |
| 20.0 | 80.0 |
| 20.1 | 0.0 |
| 35.0 | 0.0 |

Samples: 2JZ130723P16 (100 mg/vial, 100% DMSO) (see e.g., FIG. 26).

Preparation: the final product was reconstituted with Diluent, and then a solution at 2 mg/ml Bendamustine HCl in Diluent was prepared. See TABLE 15 for results.

TABLE 15
Results of DMSO content analysis in 2JZ130723P16 (100 mg/
vial).

| Sample ID | Inj # | Area | Avg | mg DSMO/vial |
|---|---|---|---|---|
| Prep 1 | 1 | 66.63255 | 6.639500 | 8.1 |
| | 2 | 66.64645 | | |

### Example 17

### Bulk Solution Hold Time Study B

A bulk solution stability comparison study was carried out by preparing both the Treanda® (RLD) formulation (prepared as described in U.S. Pat. No. 8,436,190) and a DMSO-containing test formulation at identical API/Mannitol concentrations. This Example 17 provides total impurity content over time (0, 6, 24, 48 hours) at 25° C., while similar Example 11 provides total impurity content after 0 and 72 hours.

A summary of the two lab formulations is provided in TABLE 16.

TABLE 16
Formulations for Bulk Solution Hold Time Study.

| | | Treanda ® | Test |
|---|---|---|---|
| | | Formulation | Formulation |
| | | (Lot No. | (Lot No. |
| | | 6IB130813-1) | 6IB130813-2) |
| Bulk | Bendamustine | 15 mg/ml | 15 mg/ml |
| Solution | Hydrochloride | | |
| Composition | Mannitol, USP | 25.5 mg/ml | 25.5 mg/ml |
| | q.s. to 1 ml | 30% TBA¹ v/v in Water | DMSO² |
| Processing | Compounding | 25° C. | 25° C. |
| Conditions | Temperature | | |
| | Hold at 25° C. | Yes | Yes |
| | for 48 hrs | | |

¹TBA = tert-Butyl Alcohol
²DMSO = Dimethyl Sulfoxide

For both of these lab batches, impurity content was determined periodically throughout the 25° C. hold period. The total impurities data is provided in TABLE 17.

TABLE 17
Total impurities (%) over time at 25° C.

| | Treanda ® | Test |
|---|---|---|
| Hold Time | Formulation | Formulation |
| at 25° C. | Total Imp. % | Total Imp. % |
| 0 hrs | 0.18 | 0.16 |
| 6 hrs | 0.64 | 0.17 |
| 24 hrs | 2.00 | 0.18 |
| 48 hrs | 3.68 | 0.17 |

While both formulations demonstrated comparable total impurity levels immediately after compounding (T=0), only the DMSO-containing test formulation was found to be stable with no increase in total degradation products over the 48-hour hold period. On the other hand, the RLD formulation bulk showed a significant increase in total degradation products at 25° C., even after 6 hrs (0.64%; 6 hrs typical compounding time require for commercial batch manufacturing).

Compared to the Treanda® formulation, the enhanced stability of the DMSO-containing test formulation was attributed to the elimination of water as a formula ingredient in the bulk solution. Consequently, the 48-hour stability at room temperature provides greater manufacturing flexibility than the Treanda® formulation, which must be maintained at low temperatures and has a limited hold time.

### Example 18

### Optimization Study B

The following Example describes the optimization of a bendamustine composition to increase hold time. This Example 18 provides total impurities of 80%, 90%, and 100% DMSO-containing formulations over time (0, 6, 24, 48 hours), while similar Example 12 provides total impurities of 100% DMSO-containing formulations over time (0 and 72 hours).

While the bulk hold time study B was carried at a concentration similar to the RLD product (see Example 17), the test formulation was further optimized to reduce the DMSO content in the bulk by diluting the DMSO with water or increasing the concentrations of API and Mannitol. A series of lab batches were formulated as shown in TABLE 18.

TABLE 18
Total Impurities for DMSO-containing formulations.

| | Test Formulation | Test Formulation | Test Formulation |
|---|---|---|---|
| Bulk Solution | (Lot No. | (Lot No. | (Lot No. |
| Composition | 2JZ130813P69) | 2JZ130813P70) | 2JZ130813P71) |
| Bendamustine | 25 mg/ml | 25 mg/ml | 25 mg/ml |
| Hydrochloride | | | |
| Mannitol, USP | 42.5 mg/ml | 42.5 mg/ml | 42.5 mg/ml |
| q.s. to 1 ml | 100% v/v DMSO | 90% v/v | 80% v/v |
| | | DMSO in | DMSO in |
| | | Water | Water |

| Hold Time at | | | |
|---|---|---|---|
| 25° C. | Total Imp. % | Total Imp. % | Total Imp. % |
| 0 hrs | 0.17 | N/A | N/A |
| 6 hrs | N/A | 0.19 | 0.21 |
| 24 hrs | 0.17 | 0.19 | 0.22 |
| 48 hrs | 0.18 | 0.18 | 0.25 |

The modified lab batches were compounded and held at 25° C. for 48 hours to confirm that higher percentages of water in the bulk solution or higher bulk solution concentrations did not impact degradation.

Impurity data confirmed that higher bulk solution concentrations did not impact degradation levels at all, even after the 48-hour hold period. Diluting the DMSO with 20% v/v water had a small but measurable impact on the degradation levels after the 48-hour hold period (0.25%). However, all tested DMSO-containing formulations (100%, 90%, 80% DMSO) still contained significantly less degradation levels than the Treanda® formulation (3.68% after 48 hours at 25° C.).

High bulk solution concentrations are desirable because they reduce the overall DMSO fill volume to be removed via lyophilization. Additionally, supplementing low percentages of water (0-20% v/v) into the bulk solution further reduces the volume of DMSO. As DMSO can be challenging to remove by lyophilization, these modifications can result in shorter cycle times and reduced operating costs, as well as added safety benefits due to potentially lower residual solvents present in the finished product.

### Example 19

### Lyophilization and Reconstitution

Samples were according to Example 18 unless otherwise specified.

Approximately 6 hours after compounding, portions of each lab batch were filled into the appropriate amber glass vials and lyophilized according to the cycle presented in TABLE 19.

TABLE 19
A lyophilization Cycle for Bendamustine HCl for Injection
(100 mg/vial and 25 mg/vial)

| | Rate or Hold | Temp | Time | Vacuum |
|---|---|---|---|---|
| Step | Step | (° C.) | (hr) | (mT) |
| 1. Loading | Hold | 5 | — | — |
| 2. Freezing | Rate | −45 | 1.000 | — |
| 3. Freezing | Hold | −45 | 3.000 | — |
| 4. Annealing | Rate | −20 | 0.500 | — |
| 5. Annealing | Hold | −20 | 2.000 | — |
| 6. Freezing | Rate | −45 | 0.500 | — |
| 7. Freezing | Hold | −45 | 2.000 | — |
| 8. Primary drying | Rate | −40 | 1.000 | 50 |
| 9. Primary drying | Hold | −40 | 2.000 | 38 |
| 10. Primary drying | Rate | −0 | 0.500 | 38 |
| 11. Primary drying | Hold | −0 | 24.000 | 38 |
| 12. Primary drying | Rate | 10 | 0.500 | 38 |
| 13. Primary drying | Hold | 10 | 24.000 | 38 |
| 14. Secondary drying | Rate | 40 | 3.000 | 38 |
| 15. Secondary drying | Hold | 40 | 8.000 | 38 |
| 16. Secondary drying | Rate | 25 | 1.000 | 38 |
| Total Cycle Time (hr) | — | — | 73.000 | — |

Following lyophilization, the samples contained about 100 mg/vial Bendamustine HCl, about 170 mg/vial Mannitol, and trace amounts of water and DMSO or TBA. One of the lab batches, lot number 2JZ130813P69, was additionally filled to mimic the approved low strength-25 mg/vial Bendamustine HCl, about 42.5 mg/vial Mannitol, and trace amounts of water and DMSO (lot number 2JZ130813P69-25). The physical and chemical properties for the lyophilized lab batches are summarized in TABLE 20.

TABLE 20
Total impurities, reconstitution time, residual water content,
and residual DMSO content.

| | Treanda ® | Test | | | | Test |
|---|---|---|---|---|---|---|
| | Formulation | Formulation | Test | Test | Test | Formulati [symbol] |
| Analytical | (6IB130813- | (6IB130813- | Formulation | Formulation | Formulation | (2JZ130813 [symbol] |
| Test | 1) | 2) | (2JZ130813P69) | (2JZ130813P71) | (2JZ130813P70) | 25) |
| Strength/Bulk | 100 mg/vial | 100 mg/vial | 100 mg/vial | 100 mg/vial | 100 mg/vial | 25 mg/vi [symbol] |
| Solution | TBA | 100% | 100% DMSO | 90% v/v DMSO | 80% v/v DMSO | 100% DM [symbol] |
| Solvent | | DMSO | | in Water | in Water | |
| Appearance | A white to | A white to | A white to off- | A white to off- | A white to off- | A white to [symbol] |
| | off-white | off-white | white cake | white cake | white cake | white cak [symbol] |
| | powder | cake | | | | |
| % Total | 0.78 | 0.17 | 0.20 | 0.20 | 0.20 | N/A |
| Impurities | | | | | | |
| Reconstitution | 20 | 15 | 48 | 29 | 24 | 10 |
| Time (s) | | | | | | |
| Residual | 1.7 | 1.2 | 1.1 | 1.5 | 1.4 | 1.2 |
| Water | | | | | | |
| Content (%) | | | | | | |
| Residual | N/A | 6.4 | 6.8 | 5.4 | 4.9 | 1.2 |
| DMSO | | | | | | |
| Content | | | | | | |
| (mg/vial) | | | | | | |

[symbol]

indicates data missing or illegible when filed

Compared to impurity levels of the analogous bulk solutions after a 6-hour hold, the lyophilization cycle did not impact the purity of the resulting products. Furthermore, all DMSO-containing test formulations contained lower total degradation products than the Treanda® formulation.

There was no significant difference in reconstitution time for any sample in the study, probably because all samples utilized API from the same manufacturer.

Residual water content was comparable for all samples, and may trend with the amount of water in the pre-lyophilization bulk solution.

Lastly, residual DMSO content for all samples was acceptable as compared to the 50 mg per day limit established in ICH Q3C. Diluting the bulk solution DMSO with water resulted in lower residual DMSO, as did reducing the fill volume from 4 ml (2JZ130813P69) to 1 ml (2JZ130813P69-25).

### Example 20

### Stability Data for Bendamustine/100% DMSO Formulation

The following Example describes the stability of a Bendamustine formulation with 100% DMSO at 25 mg/ml. This formulation is equivalent to the formulation described in TABLE 20 (2JZ130813P69).

A 25 mg/ml bulk solution of lab batch 3JZ140106 was prepared according to the following: 17.0499 g Mannitol was added to a 1 L glass beaker containing 320 ml of 100% DMSO. The contents were stirred with a stir bar at room temperature and 350 rpm for 10 min to fully dissolve the Mannitol. 10.0927 g Bendamustine HCl was added to the beaker and the weighing boat was rinsed into the beaker with 20 ml DMSO. The contents were stirred with a stir bar at room temperature and 350 rpm for 5 minutes to fully dissolve the API. The bulk solution was then transferred into a 400 ml volumetric flask and diluted to volume with 100% DMSO then mixed.

The formulated bulk solution was then filled into the selected container closures to mimic the approved strengths of RLD (i.e., 1.0 ml fill volume≈25 mg/vial after lyophilization; 4.0 ml fill volume≈100 mg/vial after lyophilization). The filled units were lyophilized according to the conditions presented in TABLE 19. The resulting final products are equivalent to the batches described in TABLE 20 (2JZ130813P69 and 2JZ130813P69-1).

The lyophilized final product was stored at 40° C./75% Relative Humidity (R.H.) and periodically sampled for testing. The accelerated storage conditions and test duration (6 months) were intentionally selected as they can be used to simulate expected physical and chemical behavior following 24-month storage (typical drug product shelf-life) at labeled 25° C. storage conditions.

The summarized data is shown in TABLE 21 and TABLE 22. The assay remained consistent and on-target for all time-points. The impurities grew over time, but the totals and the chromatographic profile were comparable to the RLD. The residual DMSO content trended downward slightly throughout stability testing, and all values were acceptable as compared to the 50 mg per day limit established in ICH Q3C. The reconstitution time varied during stability testing, but all results were well below the 5 minutes allowed as per the RLD package insert. Lastly, the pH measured from the reconstituted solutions were consistent and on-target for all time-points. Overall, the 6 month stability data of these final products was favorable when compared to the RLD and demonstrated the robust nature of the formulation described herein.

TABLE 21
Total impurities, DMSO (mg/vial), residual water content
(%), reconstitution time, and pH.

| | 3JZ140106-2 | 3JZ140106-2 | 3JZ140106-2 | | | Treanda RLD |
|---|---|---|---|---|---|---|
| | (25 mg/vial; | (25 mg/vial; 1 M | (25 mg/vial; 2 M | 3JZ140106-2 | 3JZ140106-2 | TB30812, exp. March 2015 |
| Sample | Initial) | 40 C.) | 40 C.) | (25 mg/vial; 3 M 40 C.) | (25 mg/vial; 6 M 40 C.) | 25 mg/vial, 3 M 40 C. |
| Impurity RRT | % Imp | % Imp | % Imp | % Imp | % Imp | % Imp |
| 0.11 | | | | | | 0.03 |
| 0.15 | | | | | 0.01 | 0.01 |
| 0.17 | | 0.01 | 0.02 | 0.02 | 0.01 | 0.05 |
| 0.18 | | 0.02 | 0.02 | 0.03 | 0.03 | 0.05 |
| 0.22 | | | 0.01 | 0.02 | 0.03 | 0.04 |
| 0.33 | 0.05 | 0.07 | 0.08 | 0.10 | 0.11 | 0.22 |
| 0.40 | 0.02 | 0.04 | 0.04 | 0.04 | 0.04 | 0.09 |
| 0.68 | | 0.01 | 0.01 | 0.02 | 0.02 | 0.02 |
| 0.71 | 0.01 | 0.05 | 0.07 | 0.10 | 0.13 | 0.11 |
| 0.79 | 0.01 | 0.01 | 0.01 | 0.01 | 0.01 | |
| 1.08 | 0.01 | 0.01 | 0.02 | 0.01 | 0.01 | 0.02 |
| 1.16 | | | | | 0.01 | |
| 1.19 | 0.01 | 0.01 | | 0.01 | 0.01 | |
| 1.21 | 0.07 | 0.08 | 0.09 | 0.09 | 0.08 | 0.03 |
| 1.22 | 0.01 | 0.01 | | | | 0.02 |
| 1.30 | | | | | | 0.08 |
| 1.34 | 0.01 | 0.12 | 0.14 | 0.15 | 0.16 | 0.23 |
| Total | 0.20 | 0.44 | 0.51 | 0.61 | 0.66 | 0.97 |
| Assay (% L.C.) | 98.8 | 100.1 | 100.1 | 100.0 | 100.1 | — |
| DMSO | 0.8 | 0.2 | 0.2 | 0.2 | 0.2 | — |
| (mg/vial) | | | | | | |
| Water (%) | 0.6 | 0.1 | 0.1 | 0.2 | 0.2 | 0.5 |
| Recon Time (s) | 10 | 20 | 25 | 25 | 30 | 50/150 |
| pH | 3.1 | 3.1 | 2.9 | 3.0 | 3.1 | 3.0 |

Bendamustine formulations (batch 3JZ140106-1) were prepared at 100 mg/vial and compared at different molar concentrations. Results are reported in TABLE 22.

TABLE 22
Total impurities, reconstitution time, residual water content,
and residual DMSO content.

| | 3JZ140106-1 | | | | |
|---|---|---|---|---|---|
| | (100 mg/vial; | 3JZ140106-1 | 3JZ140106-1 | 3JZ140106-1 | 3JZ140106-1 |
| Sample | Initial) | (100 mg/vial; 1 M 40 C.) | (100 mg/vial; 2 M 40 C.) | (100 mg/vial; 3 M 40 C.) | (100 mg/vial; 6 M 40 C.) |
| Impurity RRT | % Imp | % Imp | % Imp | % Imp | % Imp |
| 0.15 | | | | | 0.02 |
| 0.17 | | 0.01 | 0.01 | 0.01 | 0.01 |
| 0.18 | | 0.01 | 0.02 | 0.03 | 0.02 |
| 0.22 | | | 0.01 | 0.03 | 0.06 |
| 0.30 | | | 0.01 | | |
| 0.33 | 0.06 | 0.08 | 0.11 | 0.14 | 0.14 |
| 0.40 | 0.02 | 0.03 | 0.03 | 0.04 | 0.03 |
| 0.68 | | 0.01 | 0.01 | 0.02 | 0.03 |
| 0.71 | 0.01 | 0.07 | 0.09 | 0.12 | 0.16 |
| 0.79 | 0.01 | 0.01 | 0.01 | 0.01 | 0.01 |
| 1.08 | 0.02 | 0.01 | 0.01 | 0.02 | 0.01 |
| 1.09 | | | | 0.01 | |
| 1.16 | | | | | 0.01 |
| 1.19 | 0.01 | 0.01 | 0.01 | 0.01 | 0.01 |
| 1.21 | 0.07 | 0.10 | 0.11 | 0.11 | 0.10 |
| 1.22 | 0.01 | | | | |
| 1.30 | | | | | 0.03 |
| 1.34 | 0.01 | 0.15 | 0.14 | 0.17 | 0.16 |
| Total | 0.22 | 0.51 | 0.59 | 0.71 | 0.80 |
| Assay (% L.C.) | 98.7 | 100.3 | 100.3 | 100.5 | 101.7 |
| DMSO | 4.0 | 2.5 | 2.1 | 1.8 | 1.0 |
| (mg/vial) | | | | | |
| Water (%) | 0.3 | 0.2 | 0.2 | 0.2 | 0.3 |
| Recon Time (s) | 20 | 60 | 60 | 60 | 40 |
| pH | 3.1 | 3.1 | 3.0 | 3.0 | 3.0 |

Impurity Analysis.

Mobile Phase A: 10 mM Potassium Hexafluorophosphate buffer, pH 3.0. Mobile Phase B: HPLC-grade Acetonitrile. Diluent: A 60:40 mixture of Mobile Phase A and Mobile Phase B. Impurity standard: Bendamustine HCl was exposed for 1 hour at room temperature under bench to achieve H₂O saturation. A solution of 2 μg/ml Bendamustine HCl in Diluent was prepared. Sample Preparation: the final product was reconstituted with Diluent, and then a 200 μg/ml solution of Bendamustine HCl in Diluent was prepared.

The LC protocol is as follows:

Instrument: Dionex UltiMate 3000 equipped with a UV Detector and cooled Autosampler. Column: Shim-pack VP-ODS 250 L×4.6 mm, 5 μm. Flow Rate: about 1.0 ml/minute. Detector Wavelength: 233 nm, 8 nm bandwidth. Injection volume: 25 μl. Column Temperature: 25° C. Autosampler Tray Temperature: 5° C.

TABLE 23
Gradient

| Time [min] | Mobile Phase B (%) |
|---|---|
| 0.0 | 30.0 |
| 15.0 | 30.0 |
| 40.0 | 50.0 |
| 50.0 | 50.0 |
| 50.1 | 90.0 |
| 60.0 | 90.0 |
| 60.1 | 30.0 |
| 70.0 | 30.0 |

Assay Analysis.

Mobile Phase A: 10 mM Potassium Hexafluorophosphate buffer, pH 3.0. Mobile Phase B: HPLC-grade Acetonitrile. Diluent: a 60:40 mixture of Mobile Phase A and Mobile Phase B.

Assay standard: Bendamustine HCl was exposed for 1 hour at room temperature under bench to achieve H₂O saturation. A solution of 10 μg/ml Bendamustine HCl in Diluent was prepared.

Sample Preparation: the final product was reconstituted with Diluent, and then a solution of 10 μg/ml final product in Diluent was prepared.

The LC protocol is as follows:

Instrument: Dionex UltiMate 3000 equipped with a UV Detector and cooled Autosampler. Column: Shim-pack VP-ODS 250 L×4.6 mm, 5 μm. Flow Rate: about 1.0 ml/minute. Detector Wavelength: 233 nm, 8 nm bandwidth. Injection volume: 25 μl. Column Temperature: 25° C. Autosampler Tray Temperature: 5° C. Gradient: Isocratic at 50/50 Mobile Phase A:Mobile Phase B (10 minute run time).

Residual DMSO Content Analysis.

Mobile Phase A: 10 mM Potassium Dihydrogen Orthophosphate buffer. Mobile Phase B: HPLC-grade Acetonitrile. Diluent: 10 mM Disodium Hydrogen Orthophosphate buffer, pH 6.5. DMSO standard: Prepare a solution at 0.2 mg/ml DMSO in Diluent.

The LC protocol is as follows:

Instrument: Dionex UltiMate 3000 equipped with a UV Detector and cooled Autosampler. Column: Intertsil ODS-3, 5 μm, 4.6×250 mm. Flow Rate: about 0.5 ml/minute. Detector Wavelength: 210 nm, 8 nm bandwidth. Injection volume: 20 μl. Column Temperature: 30° C. Autosampler Tray Temperature: 5° C.

TABLE 24
Gradient

| Time [min] | Mobile Phase B (%) |
|---|---|
| 0.0 | 0.0 |
| 10.0 | 0.0 |
| 10.1 | 80.0 |
| 20.0 | 80.0 |
| 20.1 | 0.0 |
| 35.0 | 0.0 |

Reconstitution time.

The reconstitution for the 25 mg/vial configuration was performed by injecting 5 ml of MilliQ water with a syringe/needle 18 g 1½ and shaken by hand until the cake was completely dissolved.

The reconstitution for the 100 mg/vial configuration was performed by injecting 20 ml of MilliQ water with a syringe/needle 18 g 1½ and shaken by hand until the cake was completely dissolved.

The pH was measured directly from the reconstituted solutions prepared in as described above.

1. A pre-lyophilization solution comprising:
bendamustine or a pharmaceutically acceptable salt thereof;
a pharmaceutically acceptable excipient; and
an organic solvent comprising dimethyl sulfoxide (DMSO);
wherein the organic solvent comprises about 30% to about 100% vol/vol of the pre-lyophilization solution.

2. The solution of claim 1, wherein the pre-lyophilization solution comprises:
about 80% to about 100% dimethyl sulfoxide (DMSO); or
about 100% dimethyl sulfoxide (DMSO).

3. (canceled)

4. The solution of claim 1, wherein
the bendamustine is present at a concentration of about 1 mg/ml to about 100 mg/ml; or
the bendamustine is present at a concentration of about 2 mg/ml to about 50 mg/ml.

5. (canceled)

6. The solution of claim 1, wherein the bendamustine is present at a concentration of about 2 mg/ml, about 15 mg/ml, about 25 mg/ml, or about 50 mg/ml.

7. The solution of claim 1, wherein the bendamustine comprises a Form 2 bendamustine polymorph.

8. The solution of claim 1, wherein the pharmaceutically acceptable excipient comprises one or more of sodium phosphate, potassium phosphate, citric acid, tartaric acid, gelatin, glycine, and carbohydrates such as lactose, sucrose, maltose, mannitol, glycerin, dextrose, dextran, trehalose, or hetastarch or a mixture thereof.

9. The solution of claim 1, wherein the pharmaceutically acceptable excipient comprises mannitol.

10. The solution of claim 1, wherein the pharmaceutically acceptable excipient comprises:
mannitol at a concentration of about 1 mg/ml to about 100 mg/ml; or
mannitol at a concentration of about 25.5 mg/ml, about 42.5 mg/ml, or about 85 mg/ml.

11. (canceled)

12. The solution of claim 1, wherein the pharmaceutically acceptable salt is selected from the group consisting of 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; hydrobromic acid; hydrochloric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; nitric acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid; or a mixture thereof.

13. The solution of claim 1, wherein the pharmaceutically acceptable salt is HCl.

14. The solution of claim 1, wherein the organic solvent does not comprise tert-butyl alcohol.

15. A method for preparing a solution of claim 1, the method comprising combining the bendamustine or a pharmaceutically acceptable salt thereof, a pharmaceutically acceptable excipient, and the organic solvent.

16. A method of compounding a lyophilized bendamustine pharmaceutical composition, the method comprising:
combining bendamustine or a pharmaceutically acceptable salt thereof, a pharmaceutically acceptable excipient, and an organic solvent to form the solution of claim 1; and
lyophilizing the solution to form a lyophilized bendamustine pharmaceutical composition.

17. The method of claim 16, wherein the lyophilized bendamustine composition comprises
water at a concentration of about 0% to about 20% by weight; or
water at a concentration of about 0%, about 0.1%, about 0.2%, about 0.3%, about 0.4%, about 0.5%, about 0.6%, about 0.7%, about 0.8% about 0.9%, about 1.0%, about 1.1%, about 1.2%, about 1.3%, about 1.4%, about 1.5%, about 1.6%, about 1.7%, about 1.8%, about 1.9%, or about 2% by weight.

18. (canceled)

19. The method of claim 16, further comprising:
reconstituting the lyophilized bendamustine composition for an amount of time sufficient to form a bendamustine pharmaceutical composition suitable for administration to a subject.

20. The method of claim 19, wherein the time required for reconstitution is less than about 300 seconds, less than about 270 seconds, less than about 240 seconds, less than about 210 seconds, less than about 180 seconds, less than about 150 seconds, less than about 120 seconds, less than about 90 seconds, less than about 60 seconds, less than about 50 seconds, less than about 40 seconds, less than about 30 seconds, less than about 25 seconds, less than about 20 seconds, less than about 15 seconds, or less than about 10 seconds.

21. The method of claim 16, wherein the lyophilized bendamustine composition or the pre-lyophilization solution comprises less than about 8% bendamustine-related impurities.

22. The method of claim 16, wherein the lyophilized bendamustine composition or the pre-lyophilization solution comprises less than about 1.0%, less than about 0.9%, less than about 0.8%, less than about 0.7%, less than about 0.6%, less than about 0.5%, less than about 0.4%, less than about 0.3%, less than about 0.25%, less than about 0.2%, less than about 0.15%, less than about 0.1%, or less than about 0.05% bendamustine-related impurities.

23. A pharmaceutical composition comprising bendamustine prepared according to the method of claim 16.

24. The pharmaceutical composition of claim 23, wherein the composition comprises less than 1% impurities.
